(19) United States
(12) Patent Application Publication
(10) Pub. No.: US 2024/0390050 A1
(43) Pub. Date: Nov. 28, 2024

(54) SYSTEMS AND METHODS FOR GUIDED MANIPULATION OF THE SHAPE OF A SURGICAL IMPLANT

(71) Applicant: SEASPINE INC., CARLSBAD, CA (US)

(72) Inventors: Adrian MARIAMPILLAI, NORTH YORK (CA); Shaeffer BANNIGAN, CARLSBAD, CA (US); Jeff RICHARDSON, NORTH YORK (CA); Sonja PEJCIC, NORTH YORK (CA)

(21) Appl. No.: 18/694,261

(22) PCT Filed: Sep. 15, 2022
(86) PCT No.: PCT/US22/43639

§ 371 Date: Mar. 21, 2024

Related U.S. Application Data
(60) Provisional application No. 63/246,678, filed on Sep. 21, 2021.

(51) Int. Cl. A61B 17/88; A61B 17/56; A61B 34/20

(52) U.S. Cl. CPC A61B 17/8863; A61B 2017/568; A61B 2034/2055

(57) ABSTRACT

Systems and methods are disclosed that provide guidance for controlling the manipulation of a surgical rod according to a target rod shape. In some embodiments, tracked reference frames are secured to the surgical rod and a change in the relative orientation between the first and second trackable reference frames, measured using a tracking system, is employed to provide bend angle feedback suitable for determining when a bend formed by a rod bender conforms to a prescribed bend angle. In some embodiments, the tracked orientation of a rod bender and the tracked orientation of at least one trackable reference frame secured to the surgical rod are employed to provide feedback for controlling the roll angle of the rod bender. An augmented reality overlay showing the target rod contour may be generated based on the positions and orientations of one or both of the tracked reference frames secured to the surgical rod.

### CROSS-REFERENCE TO RELATED APPLICATION

This application claims priority to U.S. Provisional Patent Application No. 63/246,678, titled “SYSTEMS AND METHODS FOR GUIDED MANIPULATION OF THE SHAPE OF A SURGICAL IMPLANT” and filed on Sep. 21, 2021, the entire contents of which is incorporated herein by reference.

### BACKGROUND

The present disclosure relates to the manipulation of the shape of surgical implants. More particularly, the present disclosure relates to devices and methods for manipulating the shape of surgical rods for use in the correction of spinal disorders.

Neural impingement, instability and deformity are common reasons why spine surgery may be indicated. However, simply addressing these issues without restoring or maintaining global sagittal and coronal balance may lead to unsatisfactory longer-term results. During these procedures, implants may be used to help restore balance and stability to the spine, these implants may include interbody, screws and rods. Proper size, shape and positioning of implants is critical to the overall success of the procedure. Rod contouring involves shaping one or more rods to either modify the spinal balance or to lock in a particular correction achieved through the use of other implants. Studies have shown that there is large variability in a surgeons ability to bend rods to a shape that is optimal for the correction and thus it is still considered to be an art by many surgeons. The rod shape is determined by the pre-operative plan, the intraoperative assessment of the mobility of the spine, the amount of correction needed and the surgeons experience. Even once a rod shape is determined, the ability to contour the rod to that exact shape is still a challenging task.

### SUMMARY

Systems and methods are disclosed that provide guidance for controlling the manipulation of a surgical rod according to a target rod shape. In some embodiments, tracked reference frames are secured to the surgical rod and a change in the relative orientation between the first and second trackable reference frames, measured using a tracking system, is employed to provide bend angle feedback suitable for determining when a bend formed by a rod bender conforms to a prescribed bend angle. In some embodiments, the tracked orientation of a rod bender and the tracked orientation of at least one trackable reference frame secured to the surgical rod are employed to provide feedback for controlling the roll angle of the rod bender. An augmented reality overlay showing the target rod contour may be generated based on the positions and orientations of one or both of the tracked reference frames secured to the surgical rod.

Accordingly, in a first aspect, there is provided a method of providing guidance for positioning and actuating a rod bender to bend a surgical rod according to a target rod contour, the method comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track:
   an orientation of a first trackable reference frame secured to the surgical rod at a first location on the surgical rod; and
   an orientation of a second trackable reference frame secured to the surgical rod at a second location, the second location being remote from the first location such that the prescribed bend locations reside between the first location and the second location on the surgical rod; and
providing guidance for forming at least one bend of the set of bends by:
   employing the tracked orientation of the first trackable reference frame and the orientation of the second trackable reference frame to determine a measured bend angle of the surgical rod during or after bending of the surgical rod by the rod bender; and
   employing the measured bend angle to provide bend angle feedback suitable for determining when the surgical rod has been bent to the prescribed bend angle associated with the bend.

In some example implementations of the method, the positioning information is provided sequentially for forming the bends unidirectionally between the first location and the second location, and wherein each prescribed bend location is determined along an unbent portion of the surgical rod extending to the second location.

In some example implementations of the method, the positioning information is provided in a manner that facilitates the formation of the bends in an operator-selectable order, and wherein known bending characteristics of the rod bender are employed to determine a suitable bend location for an intermediate bend that is formed between two previously formed bends.

In some example implementations of the method, the rod bender is trackable by the tracking system, and wherein the positioning information is provided by:
employing the tracking system to track a position of the rod bender; and
employing the tracked position the rod bender and the tracked position and orientation of the first trackable reference frame and the second trackable reference frame to provide positioning feedback suitable for determining when the rod bender is positioned at the prescribed bend location.

In some example implementations of the method, a third trackable reference frame is secured to the rod bender, and wherein the bend parameters comprise prescribed roll angles corresponding to the set of bends, the method further comprising:
employing the tracking system to detect a roll angle of the rod bender; and
providing guidance for orienting the rod bender relative to the surgical rod to form at least one bend of the set of bends at a prescribed roll angle associated with the bend by:
   employing the roll angle of the rod bender and the orientation of one or both of the first trackable reference frame and the second trackable reference frame to provide roll angle feedback suitable for determining when the surgical rod has been oriented to form a bend at the prescribed roll angle associated with the bend.

In some example implementations, the method further comprises: employing the tracked position and orientation of the first trackable reference frame to generate real-time augmented reality images comprising a target rod contour having a first end pinned to the first location and an orientation determined based on the first trackable reference frame; displaying the augmented reality images; and updating the augmented reality images, during bending of the surgical rod, based on the tracked position and orientation of the first trackable reference frame, thereby providing augmented-reality-based visual guidance for bending the surgical rod according to the target rod contour.

The augmented reality images may be generated such that the first end of the target rod contour is tangentially aligned with the surgical rod. The rod bender may be trackable by the tracking system, wherein the positioning information is provided by: employing the tracking system to track a position of the rod bender; and employing the tracked position the rod bender and the tracked position and orientation of the first trackable reference frame and the second trackable reference frame to annotate the augmented reality images with positioning feedback suitable for determining when the rod bender is positioned at each prescribed bend location.

The positioning feedback may include generating, in the augmented reality image, an image annotation indicating a prescribed location of the rod bender for forming at least one the bends.

The method may further comprise employing the tracking system to track an orientation of the rod bender, and wherein the bend parameters comprise prescribed roll angles corresponding to the set of bends; and at each of the prescribed bend locations, employing the tracked orientation the rod bender and a detected orientation of one or both of the first trackable reference frame and the second trackable reference frame to provide orientation feedback suitable for determining when the rod bender is oriented according to a prescribed roll angle.

In another aspect, there is provided a method of providing guidance for positioning and actuating a rod bender to bend a surgical rod according to a target rod contour, the method comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod;
employing the tracking system to track a position and an orientation of the rod bender; and
providing guidance for forming at least one bend of the set of bends by:
   employing the tracked position and orientation of the rod bender to generate and display real-time augmented reality images comprising an augmented reality image annotation, the augmented reality image annotation being pinned to the tracked position of the rod bender and generated such that after the surgical rod is bent to the prescribed bend angle associated with the bend, the surgical rod spatially overlaps with the augmented reality image annotation within at least a region proximal to the bend.

In some example implementations of the method, the positioning information is provided sequentially for forming the bends unidirectionally from an initial bend location that is furthest from the first location, and wherein each prescribed bend location is determined along an unbent portion of the surgical rod extending to the first location.

In another aspect, there is provided a method of providing guidance for positioning and actuating a rod bender to bend a surgical rod according to a target rod contour, the method comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod, the surgical rod extending from the first location along a rod axis;
employing the tracked position and orientation of the first trackable reference frame to generate and display real-time augmented reality images, the real-time augmented reality images comprising augmented reality image annotations indicating the prescribed bend locations along the rod axis, such that when the bends are formed in a unidirectional sequence of bends beginning from a bend that lies at a furthest distal location relative to the first trackable reference frame, the augmented reality image annotations on a proximal side of a given bend show the locations along the remaining unbent portion of the surgical rod for forming the remaining bends; and
updating the augmented reality images, during bending of the surgical rod, based on the tracked position and orientation of the first trackable reference frame, thereby providing augmented-reality-based visual guidance for bending the surgical rod according to the target rod contour.

Th method may further comprise: employing a tracking system to track an orientation of a second trackable reference frame secured to the surgical rod at a second location, the second location being remote from the first location such that the prescribed bend location resides between the first location and the second location on the surgical rod;
employing the tracked position and orientation of the second trackable reference frame to annotate the real-time augmented reality images with a target rod contour having a first end pinned to the second location and an orientation determined based on the second trackable reference frame;
updating the augmented reality images, during bending of the surgical rod, based on the tracked position and orientation of the second trackable reference frame, thereby providing augmented-reality-based visual guidance for bending the surgical rod according to the target rod contour.

In some example implementations of the method, the augmented reality images are generated such that the first end of the target rod contour is tangentially aligned with the surgical rod.

In another aspect, there is provided a method of providing guidance for positioning and actuating a rod bender to bend a surgical rod according to a prescribed bend angle at a prescribed bend location, the method comprising:
providing positioning information suitable for positioning the rod bender at the prescribed bend location along the surgical rod;
employing a tracking system to track:
   an orientation of a first trackable reference frame secured to the surgical rod at a first location on the surgical rod; and
   an orientation of a second trackable reference frame secured to the surgical rod at a second location, the second location being remote from the first location such that the prescribed bend location resides between the first location and the second location on the surgical rod; and
employing the tracked orientation of the first trackable reference frame and the orientation of the second trackable reference frame to determine a measured bend angle of the surgical rod during or after bending of the surgical rod by the rod bender; and
employing the measured bend angle to provide bend angle feedback suitable for determining when the surgical rod has been bent to the prescribed bend angle.

In some example implementations of the method, the bend angle feedback comprises providing qualitative feedback of a deviation of the measured bend angle from the prescribed bend angle.

In some example implementations of the method, the bend angle feedback comprises providing quantitative feedback of a deviation of the measured bend angle from the prescribed bend angle.

In some example implementations of the method, the positioning information is provided as a distance relative to at least one of the first location and the second location.

In some example implementations of the method, the rod bender is trackable by the tracking system, and wherein the positioning information is provided by:
employing the tracking system to track a position of the rod bender; and
employing the tracked position the rod bender and the tracked position and orientation of at least one of the first trackable reference frame and the second trackable reference frame to provide positioning feedback suitable for determining when the rod bender is positioned at the prescribed bend location.

In some example implementations of the method, a third trackable reference frame is secured to the rod bender, the method further including: employing the tracking system to detect a roll angle of the rod bender; and employing the roll angle of the rod bender and the orientation of one or both of the first trackable reference frame and the second trackable reference frame to provide roll angle feedback suitable for determining when the surgical rod has been bent to a prescribed roll angle.

In another aspect, there is provided a method of providing guidance for positioning and actuating a rod bender to bend a surgical rod, the method comprising:
employing a tracking system to track a position and an orientation of a first trackable reference frame secured to the surgical rod at a first location;
employing the tracked position and orientation of the first trackable reference frame to generate real-time augmented reality images comprising a target rod contour having a first end pinned to the first location and an orientation determined based on the first trackable reference frame;
displaying the augmented reality images; and
updating the augmented reality images, during bending of the surgical rod, based on the tracked position and orientation of the first trackable reference frame, thereby providing augmented-reality-based visual guidance for bending the surgical rod according to the target rod contour.

In some example implementations of the method, the augmented reality image is generated such that the first end of the target rod contour is tangentially aligned with the surgical rod.

In some example implementations of the method, a trackable marker is secured to the surgical rod at a second location remote from the first location, and wherein the augmented reality images are generated such that a second end of the target rod contour resides along line extending between the first location and the second location.

In some example implementations of the method, a second trackable reference frame is secured to the surgical rod at a second location remote from the first location, and wherein the augmented reality images are generated such that a second end of the target rod contour resides along line extending between the first location and the second location.

The method may further comprise employing the orientation of the first trackable reference frame and the orientation of the second trackable reference frame to determine a measured bend angle of the surgical rod during or after bending of the surgical rod by the rod bender; and employing the measured bend angle to provide bend angle feedback. The method may further comprise employing the tracking system to track a position and an orientation of the second trackable reference frame; and providing feedback indicative of a roll of the second trackable reference frame relative to the first trackable reference frame.

In some example implementations, the method further comprises: employing the tracking system to track a trackable rod bender having an additional trackable reference frame secured thereto and determine an orientation of the rod bender; and providing bender orientation feedback indicative of a roll of the rod bender relative to the first trackable reference frame.

The target rod contour may be a first target rod contour defined within a first plane, and the bender orientation feedback may be provided to facilitate orientation of the rod bender such that a bending plane of the rod bender is parallel to the first plane during bending of the surgical rod. The augmented reality images may be first real-time augmented reality images, and the method may further comprise:
employing the position and orientation of the first trackable reference frame to generate second real-time augmented reality images comprising a second target rod contour, such that a first end of the second target rod contour is pinned to the first location, and wherein the second target rod contour resides in a second plane that is orthogonal to the first plane;
displaying the second augmented reality images; and
updating the second augmented reality images, during bending of the surgical rod in the second plane, based on the tracked position and orientation of the first trackable reference frame, thereby providing augmented-reality-based visual guidance for bending the surgical rod according to the second target rod contour;
wherein additional bender orientation feedback is provided to facilitate orientation of the rod bender such that the bending plane of the rod bender is parallel to the second plane during bending of the surgical rod.

In some example implementations, the method further comprises displaying, in a user interface configured to receive input for varying the target rod contour relative to a preoperatively-defined rod contour. The user interface may be configured such that the shape of the target rod contour variable, in response to the input, between the preoperatively-defined rod contour and an intraoperatively-determined spinal contour. The user interface may be configured such that the shape of the target rod contour is continuously variable, via a slider, between the preoperatively-defined rod contour and the intraoperatively-determined spinal contour.

In another aspect, there is provided a system for providing guidance for positioning and actuating a rod bender to bend a surgical rod according to a target rod contour, the system comprising:
a tracking system; and
control and processing circuitry operatively coupled to said tracking system, said control and processing circuitry comprising at least one processor and associated memory, the memory comprising instructions executable by said at least one processor for performing operations comprising:
   obtaining bend parameters for bending the surgical rod according to a set of bends that approximate the target rod contour, the bend parameters comprising prescribed bend locations along the surgical rod and corresponding prescribed bend angles;
   providing positioning information suitable for positioning the rod bender at the prescribed bend locations along the surgical rod;
   employing said tracking system to track:
      an orientation of a first trackable reference frame secured to the surgical rod at a first location on the surgical rod; and
      an orientation of a second trackable reference frame secured to the surgical rod at a second location, the second location being remote from the first location such that the prescribed bend locations reside between the first location and the second location on the surgical rod; and
   providing guidance for forming at least one bend of the set of bends by:
      employing the tracked orientation of the first trackable reference frame and the orientation of the second trackable reference frame to determine a measured bend angle of the surgical rod during or after bending of the surgical rod by the rod bender; and
      employing the measured bend angle to provide bend angle feedback suitable for determining when the surgical rod has been bent to the prescribed bend angle associated with the bend.

In another aspect, there is provided a system for providing guidance for positioning and actuating a rod bender to bend a surgical rod according to a target rod contour, the system comprising:
a tracking system; and
control and processing circuitry operatively coupled to said tracking system, said control and processing circuitry comprising at least one processor and associated memory, the memory comprising instructions executable by said at least one processor for performing operations comprising:
   obtaining bend parameters for bending the surgical rod according to a set of bends that approximate the target rod contour, the bend parameters comprising prescribed bend locations along the surgical rod and corresponding prescribed bend angles;
   providing positioning information suitable for positioning the rod bender at the prescribed bend locations along the surgical rod;
   employing said tracking system to track an orientation of a first trackable reference frame secured to the surgical rod at a first location on the surgical rod;
   employing said tracking system to track a position and an orientation of the rod bender; and
   providing guidance for forming at least one bend of the set of bends by:
      employing the tracked position and orientation of the rod bender to generate and display real-time augmented reality images comprising an augmented reality image annotation, the augmented reality image annotation being pinned to the tracked position of the rod bender and generated such that after the surgical rod is bent to the prescribed bend angle associated with the bend, the surgical rod spatially overlaps with the augmented reality image annotation within at least a region proximal to the bend.

In another aspect, there is provided a system for providing guidance for positioning and actuating a rod bender to bend a surgical rod according to a target rod contour, the system comprising:
a tracking system;
a display device; and
   control and processing circuitry operatively coupled to said tracking system and said display device, said control and processing circuitry comprising at least one processor and associated memory, the memory comprising instructions executable by said at least one processor for performing operations comprising:
   obtaining bend parameters for bending the surgical rod according to a set of bends that approximate the target rod contour, the bend parameters comprising prescribed bend locations along the surgical rod and corresponding prescribed bend angles;
   providing positioning information suitable for positioning the rod bender at the prescribed bend locations along the surgical rod;
   employing said tracking system to track an orientation of a first trackable reference frame secured to the surgical rod at a first location on the surgical rod, the surgical rod extending from the first location along a rod axis;
   employing the tracked position and orientation of the first trackable reference frame to generate and display on said display device real-time augmented reality images, the real-time augmented reality images comprising augmented reality image annotations indicating the prescribed bend locations along the rod axis, such that when the bends are formed in a unidirectional sequence of bends beginning from a bend that lies at a furthest distal location relative to the first trackable reference frame, the augmented reality image annotations on a proximal side of a given bend show the locations along the remaining unbent portion of the surgical rod for forming the remaining bends; and
   updating the augmented reality images, during bending of the surgical rod, based on the tracked position and orientation of the first trackable reference frame, thereby providing augmented-reality-based visual guidance for bending the surgical rod according to the target rod contour.

In another aspect, there is provided a system for providing guidance for positioning and actuating a rod bender to bend a surgical rod according to a target rod contour, the system comprising:
a tracking system;
a display device; and
control and processing circuitry operatively coupled to said tracking system and said display device, said control and processing circuitry comprising at least one processor and associated memory, the memory comprising instructions executable by said at least one processor for performing operations comprising:
   controlling said tracking system to track a position and an orientation of a first trackable reference frame secured to the surgical rod at a first location;
   employing the tracked position and orientation of the first trackable reference frame to generate real-time augmented reality images comprising a target rod contour having a first end pinned to the first location and an orientation determined based on the first trackable reference frame;
   displaying the augmented reality images on said display device; and
   updating the augmented reality images, during bending of the surgical rod, based on the tracked position and orientation of the first trackable reference frame, thereby providing augmented-reality-based visual guidance for bending the surgical rod according to the target rod contour.

In another aspect, there is provided a non-transitory computer-readable storage medium having stored therein data representing instructions executable by a processor for providing guidance for positioning and actuating a rod bender to bend a surgical rod according to a target rod contour, the storage medium comprising instructions for performing operations including:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
controlling a tracking system to track:
   an orientation of a first trackable reference frame secured to the surgical rod at a first location on the surgical rod; and
   an orientation of a second trackable reference frame secured to the surgical rod at a second location, the second location being remote from the first location such that the prescribed bend locations reside between the first location and the second location on the surgical rod; and
providing guidance for forming at least one bend of the set of bends by:
   employing the tracked orientation of the first trackable reference frame and the orientation of the second trackable reference frame to determine a measured bend angle of the surgical rod during or after bending of the surgical rod by the rod bender; and
   employing the measured bend angle to provide bend angle feedback suitable for determining when the surgical rod has been bent to the prescribed bend angle associated with the bend.

In another aspect, there is provided a non-transitory computer-readable storage medium having stored therein data representing instructions executable by a processor for providing guidance for positioning and actuating a rod bender to bend a surgical rod according to a target rod contour, the storage medium comprising instructions for performing operations including:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod;
employing said tracking system to track a position and an orientation of the rod bender; and
providing guidance for forming at least one bend of the set of bends by:
   employing the tracked position and orientation of the rod bender to generate and display real-time augmented reality images comprising an augmented reality image annotation, the augmented reality image annotation being pinned to the tracked position of the rod bender and generated such that after the surgical rod is bent to the prescribed bend angle associated with the bend, the surgical rod spatially overlaps with the augmented reality image annotation within at least a region proximal to the bend.

In another aspect, there is provided a non-transitory computer-readable storage medium having stored therein data representing instructions executable by a processor for providing guidance for positioning and actuating a rod bender to bend a surgical rod according to a target rod contour, the storage medium comprising instructions for performing operations including:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod, the surgical rod extending from the first location along a rod axis;
employing the tracked position and orientation of the first trackable reference frame to generate and display on a display device real-time augmented reality images, the real-time augmented reality images comprising augmented reality image annotations indicating the prescribed bend locations along the rod axis, such that when the bends are formed in a unidirectional sequence of bends beginning from a bend that lies at a furthest distal location relative to the first trackable reference frame, the augmented reality image annotations on a proximal side of a given bend show the locations along the remaining unbent portion of the surgical rod for forming the remaining bends; and
updating the augmented reality images, during bending of the surgical rod, based on the tracked position and orientation of the first trackable reference frame, thereby providing augmented-reality-based visual guidance for bending the surgical rod according to the target rod contour.

In another aspect, there is provided a non-transitory computer-readable storage medium having stored therein data representing instructions executable by a processor for providing guidance for positioning and actuating a rod bender to bend a surgical rod according to a target rod contour, the storage medium comprising instructions for performing operations including:
controlling a tracking system to track a position and an orientation of a first trackable reference frame secured to the surgical rod at a first location;
employing the tracked position and orientation of the first trackable reference frame to generate real-time augmented reality images comprising a target rod contour having a first end pinned to the first location and an orientation determined based on the first trackable reference frame;
displaying the augmented reality images on a display device; and
updating the augmented reality images, during bending of the surgical rod, based on the tracked position and orientation of the first trackable reference frame, thereby providing augmented-reality-based visual guidance for bending the surgical rod according to the target rod contour.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1A schematically illustrates a tracked surgical rod with prescribed bend locations indicated along the surgical rod for bending the surgical rod according to a target rod contour.

FIG. 1B illustrates the measurement of the bend angle via tracking of the relative orientation between the first and second trackable reference frames.

FIGS. 1C and 1D illustrate example bend angle feedback that provides guidance for bending the surgical rod according to a prescribed bend angle.

FIG. 1E illustrates the measurement of the bend angle of the second bend via tracking of the relative orientation between the first and second trackable reference frames.

FIG. 1F illustrates example bend angle feedback that provides guidance for bending the surgical rod according to a prescribed bend angle for a second bend.

FIGS. 2A-2E illustrate various example tracked and untracked implementations of a rod bender.

FIG. 3 schematically illustrates the use of a tracked rod bender and a tracked reference frame secured to the surgical rod to provide roll angle feedback.

FIG. 4 is a flow chart illustrating an example process of generating augmented reality annotations based on positional information detected by a tracking system.

FIGS. 5A-5K show an example user interface presenting images with an augmented reality overlay of the target rod contour.

FIGS. 6A-6C schematically illustrate the rendering of an augmented reality overlay pinned to a tracked rod bender in order to provide augmented reality guidance for forming a bend with a prescribed bend angle.

FIG. 7 illustrates an example embodiment in which augmented reality overlays corresponding to the prescribed bend locations are shown extending along an unbent portion of the surgical rod.

FIGS. 8A-8E schematically illustrate an example embodiment in which guidance for bending a surgical rod is provided in the form of an augmented reality overlay of the target rod contour, where one end of the target rod contour overlay is pinned to the location of a first trackable reference frame that is secured to the surgical rod and the other end of the target rod contour overlay is constrained to lie along a line extending between the first and second trackable reference frames.

FIG. 9 schematically illustrates an example embodiment in which guidance for bending a surgical rod is provided in the form of an augmented reality overlay of the target rod contour, where a first end of the target rod contour overlay is pinned to the location of a first trackable reference frame that is secured to the surgical rod and where the target rod contour overlay is oriented such that the first end of the target rod contour overlay is tangentially aligned with the surgical rod.

FIGS. 10A-10D illustrate example implementations of a user interface that facilitates the variation and selection of a target rod contour that resides between a pre-operatively defined target rod contour and an intraoperatively determined spinal orientation.

FIG. 11 schematically illustrates and example system for providing guidance during manipulation of a surgical rod.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Various example embodiments of the present disclosure provide methods and systems that deliver guidance facilitating the bending of a surgical rod according to a target rod contour. An initial target rod contour for achieving spinal balance or for applying a desired correction is typically determined pre-operatively when formulating a pre-operative (surgical) plan. This target rod contour may be intra-operatively refined, for example, in order to take into account differences between pre-operative and intraoperative spine configuration, and/or to account for limited mobility and flexibility that is observed intraoperatively.

In some example embodiments, feedback is provided to facilitate the bending of a surgical rod according to a prescribed set of bends having associated bending parameters, with each prescribed bend having an associated prescribed bend location and a prescribed bend angle. The target rod contour can be determined, for example, by intraoperatively selecting the position of screw heads and configuring the bends such that the rod passes through the center of each screw head, or, for example using pre-planning software to determine the suitable lordosis and/or kyphosis angle in a region of the spine.

A target rod contour can be parameterized, for example, according to user input, such as via a user interface configured to receive input defining the target rod contour in terms of lordosis and/or kyphosis angles that are each approximated by a prescribed number of bends. In some example implementations, a given target rod contour may be parameterized in terms of a set of prescribed bend locations and prescribed bend angles by subdividing the target rod contour into a set of contour segments by specifying a set of bend points, approximating each segment contour with a linear segment such that set of linear segments are connected, and calculating a prescribed bend angle for each prescribed bend location that lies between two adjacent segments. In some example implementations, the bend points may be specified to be equally spaced, spaced according to the local curvature, or spaced according to the positions associated with a surgical intervention, such as locations for the implantation of screws. It will be understood that a wide variety of methods may be employed for the selection of the bend points, and multiple methods may be employed when defining the points to parameterize a given target rod contour, such based on a combination of screw position and based on target rod curvature.

While the bend locations can be communicated to an operator and verified, for example, by measuring a distance along the surgical rod, or, for example, using a tracked position of a rod bender relative to a tracked position on the surgical rod, it can be challenging for an operator to determine how much force should be applied when forming a bend. Moreover, it can be very difficult for an operator to determine when a bend has been formed with a bend angle that will result in the appropriate shape for approximating the target rod contour. Finally, even in cases in which an appropriate amount of force has been applied to generate a prescribed bend angle, elastic deformation may result in the final bend angle being different from the prescribed bend angle.

The present inventors set out to solve this problem by employing the use of trackable reference frames secured to the surgical rod to provide direct and unambiguous feedback during or after the formation of a bend, thereby facilitating the formation of a bend that matches or sufficiently approximates a prescribed bend angle.

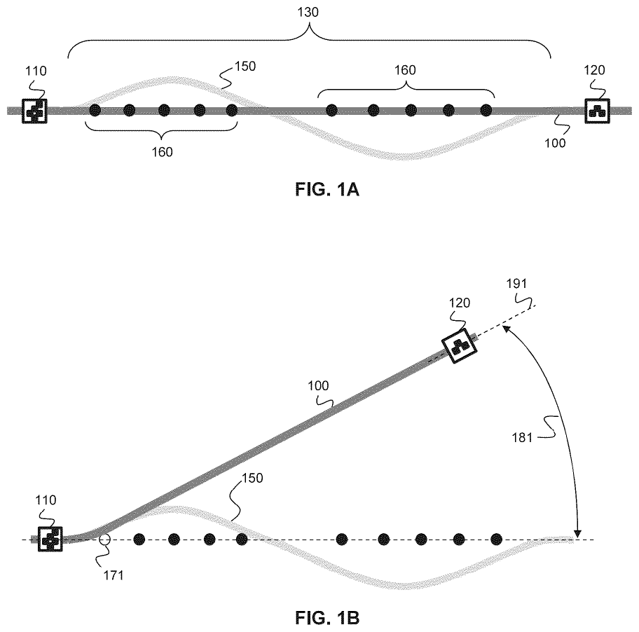

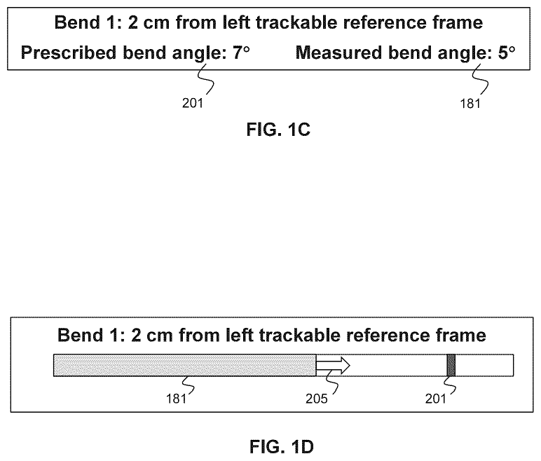

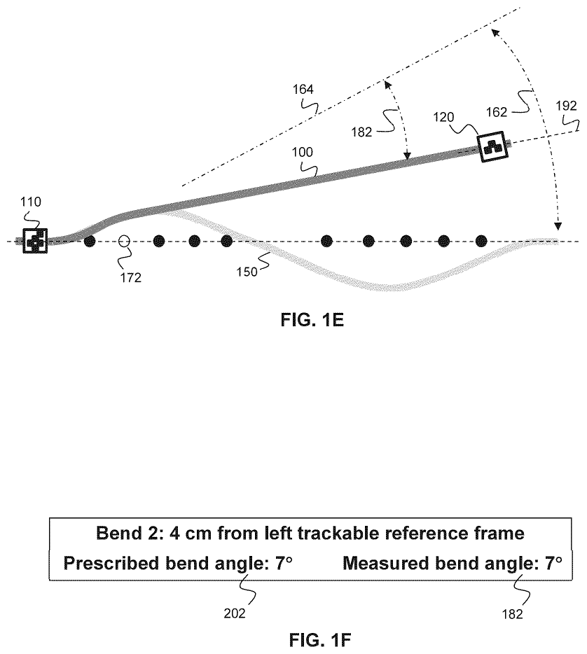

FIG. 1A illustrates an example implementation in which first and second trackable reference frames 110 and 120 are respectively secured to a surgical rod 100 prior to bending the surgical rod 100. The first trackable reference frame 110 resides at a first location on the surgical rod 100 and the second trackable reference frame 120 resides at a second location that is remote from the first location, such that the prescribed bend locations 160 reside within an intermediate region 130 between the first and second locations on the surgical rod 100. A tracking system is employed to track the real-time orientation of the trackable reference frames 110, 120 in order to provide feedback associated with the bend angle during or after the formation of a bend.

Each trackable reference frame includes at least one marker and/or structure that facilitates a determination of the three-dimensional position and orientation of the tracked reference frame, thereby facilitating a determination of the position and orientation of the local rod segment to which the trackable reference frame is secured. In one example case of an optical tracking system, three or more fiducial markers (either active or passive) may be supported on the trackable reference frame. In another example implementation, electromagnetic tracking sensors may be employed. In other example implementations, a two-dimensional barcode or glyph may be detectable by a tracking system, or a three-dimensional shape may be detectable a surface-detection-based tracking system, to facilitate the determination of the position and orientation of the trackable reference frame.

FIG. 1A also shows the superimposed target rod contour 150, indicating the desired shape of the surgical rod after bending, and the prescribed bend locations 160. Although several example implementations of the present disclosure involve the display of the target rod contour as an augmented reality image annotation, the superimposed target rod contour 150 is provided in FIG. 1A for illustrative purposes and is not intended to require the use of augmented reality. Indeed, in some example implementations, the rod bending guidance can be provided in the absence of the display of the target rod shape. Likewise, while several example implementations of the present disclosure involve the display of the prescribed bend locations as an augmented reality image annotations, the prescribed bend locations 160 are shown in FIG. 1A for illustrative purposes and are not intended to require the use of augmented reality.

In order to generate one of the bends using a rod bender, the operator is provided positioning information enabling the operator to position the rod bender the prescribed bend locations along the surgical rod. This positioning information can be provided according to a wide range of implementations. For example, the operator can be presented with quantitative distance measures indicating the locations, along the surgical rod 100, of the prescribed bend locations 160. For example, such distance measures can be displayed on a user interface, in parallel (all prescribed bend locations shown) or serially/sequentially (bend locations shown one at a time).

The distance measures can be provided relative to one or more ends of the surgical rod 100, and/or relative to one or both of the first and second locations respectively associated with the first and second trackable reference frames 110, 120.

In other example implementations, described in further detail below, the prescribed bend locations, along the surgical rod 100, can be presented in the form of augmented reality annotations that are generated based on the tracked position and orientation of one or both of the trackable reference frames 110, 120.

The positioning information enables the operator to place the rod bender at the prescribed bend locations for forming the bends. One or more of the bends may be formed using feedback generated based on the tracked orientations of the first and second trackable reference frames 110, 120. Such an example implementation is illustrated in FIGS. 1B-1F.

FIG. 1B illustrates the formation of an initial bend. The operator, having positioned the rod bender at the prescribed location 171 of the bend, of controls the rod bender to bend the surgical rod 100. As the surgical rod 100 is bent, the remaining portion of the surgical rod beyond the bend rotates. As shown in the figure, the bend angle generated by the bender corresponds to the change in the relative orientation between the first and second trackable reference frames 110, 120, as evidenced in the figure by the change in the angle between the local rod axes associated with the first and second trackable reference frames 110, 120.

The tracked change in the relative orientation of the first and second trackable reference frames 110, 120 may therefore be employed to measure the bend angle generated by actuation of the rod bender. This measured bend angle may be compared to the prescribed bend angle in order to generate feedback (e.g. real-time feedback) that the operator can employ to determine when the surgical rod 100 has been bent to the prescribed bend angle associated with the bend. The prescribed bend angle may include an additionally angle offset that is selected to compensate for partial elastic recoil of the surgical rod after the surgical rod is released by the rod bender.

For example, FIG. 1C illustrates example bend angle feedback that enables the operator to determine when the surgical rod has been bent to the prescribed bend angle associated with the first bend (the example feedback shows the prescribed bend location relative to the left trackable reference frame). The example feedback is quantitative in nature and permits a comparison between the prescribed bend angle 201 and the measured bend angle 181. In another non-limiting example of quantitative bend angle feedback, the difference between the prescribed bend angle 201 and the measured bend angle 181 may be displayed.

In other example implementations, the bend angle feedback may be provided as qualitative feedback, or as a combination of qualitative and quantitative feedback. FIG. 1D illustrates an example implementation in which the bend angle feedback is qualitative in the form of a bar that qualitatively illustrates the size of the measured bend angle 181 relative to the prescribed bend angle 201, optionally including a direction annotation 205 indicating that the bend angle should be increased in order to achieve the prescribed bend angle 201.

It will be understood that the bend angle feedback may be provided according to a wide variety of modalities, including, but not limited to, information displayed on a user interface, acoustic feedback, and haptic feedback.

FIG. 1E illustrates the tracking of the orientation of the first and second trackable reference frames 110, 120 to provide bend angle feedback during the formation of another bend at another prescribed bend location. The figure illustrates the example case in which the bend angle feedback is generated to guide the formation of the second bend at the second prescribed bend location 172. As can be seen in the figure, the bend angle 182 formed by actuation of the rod bender corresponds to the change in the relative orientation of the first and second trackable markers (e.g. as evidenced by the angle 182 between the local rod axes 191 and 192 associated with the second trackable marker 120). Accordingly, the measured bend angle 182 can be compared to the prescribed bend angle for the second bend in order to generate bend angle feedback that enables the operator to ensure that the applied bend angle sufficiently corresponds to the prescribed bend angle. FIG. 1F illustrates the generation of example bend angle feedback for forming the second bend according to the prescribed bend angle, showing the case in which the operator has employed to feedback to obtain agreement between the prescribed bend angle 202 and the measured bend angle 182.

Since the bend angle generated by the rod bender is measured based on the change in the relative orientation of the first and second trackable reference frames, the bend angle measurement is not affected by global translations and/or rotations of the surgical rod. For the same reason, the order of the generation of the bends does not affect the bend angle measurement. Furthermore, while the present example embodiment is illustrated in the case of the bending of an initially straight rod, the present method of generating bend angle feedback can be implemented using a surgical rod that is initially in a pre-bent configuration. Finally, the present example methods of providing bend angle feedback need not be implemented for all of the bends and may be implemented for only a subset of the bends, with the remaining bends being generated using another method (such as, for example, freehand bending or bending according to pre-set bend angles).

In some example implementations, the positioning information may be provided sequentially, one bend at a time, for forming the bends unidirectionally between the first location and the second location. According to such an example method, each prescribed bend location is determined along an unbent portion of the surgical rod extending to the second location. This approach avoids the need to re-calculate the positions of the prescribed bending locations during bending of the surgical rod.

In another example implementation, the positioning information may be provided in a manner that facilitates the formation of the bends in an operator-selectable order. For example, a user interface may be provided that enables the operator to select the order of generation of the bends. Such an implementation, when resulting in the formation of an intermediate bend between two or more previously-formed bends, may require the re-calculation of the prescribed location of the intermediate bend. This may be achieved, for example, using known bending characteristics of the rod bender. For example, a known radius of curvature of the bender may be employed in conjunction with the known bend angles of two or more previously-formed bends to determine a new determination of the prescribed bend location for the intermediate bend.

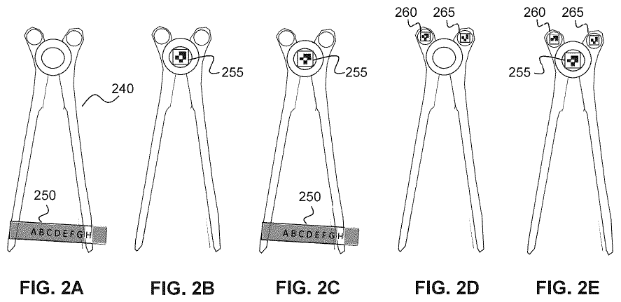

FIGS. 2A-2E illustrate various non-limiting example implementations of rod benders, including both trackable and non-trackable rod benders. FIG. 2A illustrates a non-trackable rod bender 240 that includes a caliper mechanism 250 for applying a prescribed bending angle. FIG. 2B illustrates an example rod bender that includes a trackable marker 255, which may be trackable for a determination of position or both position and orientation. FIG. 2C illustrates an example tracker rod bender that includes a trackable marker 255 and a caliper mechanism 250 for applying a prescribed bending angle, thereby facilitating actuation according to a prescribed bend angle at a prescribed bend location and orientation of the bender. FIGS. 2D and 2E illustrate an example implementation that permits tracking of the two arms of the bender via tracking markers 260 and 265, thereby facilitating tracking of the bend location, bender orientation and the bending angle, where FIG. 2E includes an additional trackable marker 255 on the hinge pin (or center post) of the rod bender.

While the present example implementations refer to handheld rod benders, it will be understood that the rod bender need not be handheld and that other types of rod benders may be employed in the alternative, such as, for example, a system that bends a rod between two dies, one of which may be actuated to control the bend angle, and in which the bend angle is visible during the bending process.

In example implementations in which the position of the rod bender is trackable, the tracked position of the rod bender and the tracked position and orientation of at least one of the first trackable reference frame and the second trackable reference frame may be employed to provide positioning feedback suitable for determining when the rod bender is positioned at the prescribed bend location.

For example, in the example case in which the bends are formed sequentially and unidirectionally, one bend at a time, between the first location and the second location, with the bend positions being prescribed relative to the second location, the position and orientation of the unbent portion of the surgical rod may be determined based on the tracked position and orientation of the trackable reference frame residing at the second location, and the tracked position of the rod bender may be employed to provide feedback suitable for determining when the rod bender resides at a prescribed bend location along the unbent portion of the surgical rod.

Alternatively, in the example case in which the bends are not formed unidirectionally along the surgical rod, the tracked positions and orientations of the first and second trackable reference frames, and known bending characteristics of the rod bender, may be employed to update the prescribed bend locations between to previously formed bends, and the updated prescribed bend locations, along with the tracked position of the rod bender, may be employed to provide feedback suitable for determining when the rod bender resides at the updated prescribed bend location along the intermediate portion of the surgical rod.

In some example implementations, the rod bender may include a trackable reference frame, thereby enabling a determination of the orientation of the rod bender. The tracked orientation of the rod bender and the tracked orientation of one or both of the first trackable reference frame and the second trackable reference frame may be employed to determine a roll angle of the rod bender.

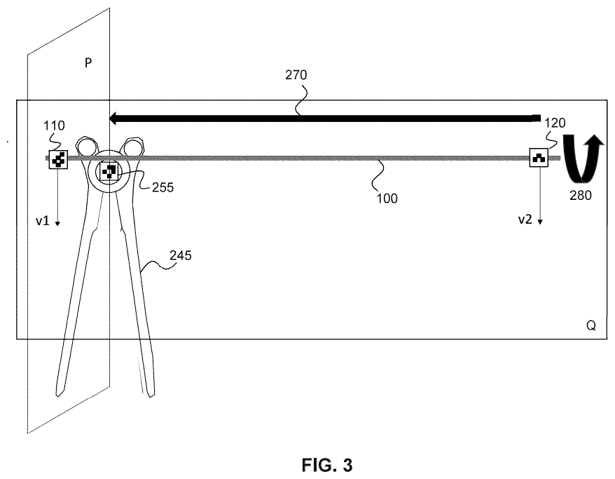

FIG. 3 schematically illustrates the use of tracked rod bender 245 to provide feedback regarding the roll of the rod bender 245 relative to the tracked surgical rod 100. As shown in the figure, the tracked rod bender 245 includes a trackable reference frame 255 that is employed the tracking system to track an orientation of the rod bender 245, relative to the orientation of one or both of the trackable reference frames 110, 120, thereby facilitating measurement of the roll angle 280 of the tracked rod bender 245 relative to a plane Q associated with the surgical rod 100 (e.g. a plane associated with one or more prescribed bends for bending the surgical rod 100 according to the target rod contour). The measured roll angle may be compared with the prescribed roll angle to provide roll angle feedback suitable for determining when the tracked rod bender 245 has been oriented with a suitable orientation to form a given bend at a prescribed roll angle. As in the preceding example embodiments, the bend angle may be measured based on the tracked relative orientation between the first and second tracked reference frames 110 and 120, e.g. the angle between orientation vectors v1 and v2 respectively associated with the first and second trackable reference frames.

Such implementations involving the tracking of the orientation of a trackable rod bender may be beneficial for providing feedback suitable for controlling the formation of a plurality of bends that cause the target rod contour to extend in three dimensions, such that the target rod contour resides beyond a two-dimensional plane. For example, referring again to FIG. 3, a first subset of the bends may have prescribed roll angles such that the bends are formed within the plane Q, while a second subset of the bends may have prescribed roll angles such that the bends are formed within the plane P, and the tracked orientation of the trackable rod bender 245 may be employed to provide roll angle feedback sufficient to guide the placement of the trackable rod bender 245 with the appropriate orientation to generate each bend with the correct roll angle.

For example, in the example case in which the bends are formed sequentially and unidirectionally, one bend at a time, between the first location and the second location, with the prescribed bend locations determined along direction 270, the roll angle feedback may be determined based on the orientation of the rod bender 245 and the tracked position and orientation of a single trackable reference frame residing at the second location. Alternatively, in example cases in which the bends are not formed unidirectionally along the surgical rod, the roll angle feedback may be generated based on the tracked orientation of the trackable rod bender 245, the tracked positions and orientations of the first and second trackable reference frames 110, 120, and known bending characteristics of the rod bender 245.

In some example embodiments, augmented reality images may be generated and displayed to provide additional feedback for forming one or more of the prescribed set of bends. For example, images may be obtained with a camera having a field of view that includes the surgical rod and the tracked position and orientation of the first trackable reference frame may be employed to generate real-time augmented reality images that include an augmented reality image annotation displaying the target rod contour, based on a known transformation between a frame of reference of the tracking system and a frame of reference of the imaging camera. The augmented reality images may be displayed, for example, on a display screen, or, for example, via tracked head-mounted display device (e.g. with device tracking and/or gaze detection). Alternatively, the augmented reality images may be generated as a heads-up display, for example, using tracked heads-up display headset (without requiring a camera). The augmented reality image annotation may be generated such that a first end of the surgical rod contour is pinned to the first location and has an orientation determined based on the first trackable reference frame.

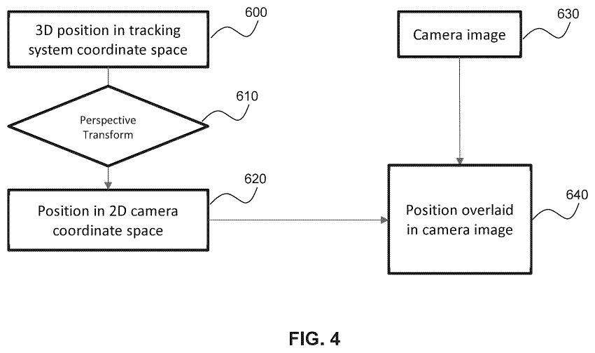

FIG. 4 illustrates an example process for generating a coordinate transform between the frame of reference of the tracking system and the frame of reference of the augmented reality display device. An example tracking system includes up of one or more cameras/sensors to track the position of one or more trackable reference frames. The tracking system outputs the position information 600 associated with the tracked reference frames. The tracking system is calibrated to the camera which outputs a 2D images 630 such that a known 3D position in the tracking system coordinate space can projected into the 2D camera image space (or, equivalently, into the space associated with a heads-up display that is absent of a camera). This transform 610 can be generally described as a perspective transform for mapping a 3D position from the reference frame of the tracking system to coordinates 620 in the 2D camera image space. It can be determined by using a set of known correspondences between points in the tracking systems 3D space and the corresponding points in the 2D image space. Once the positional information has been projected into the 2D image space it can be overlayed or rendered directly into a 2D image frame 640 for display to the user.

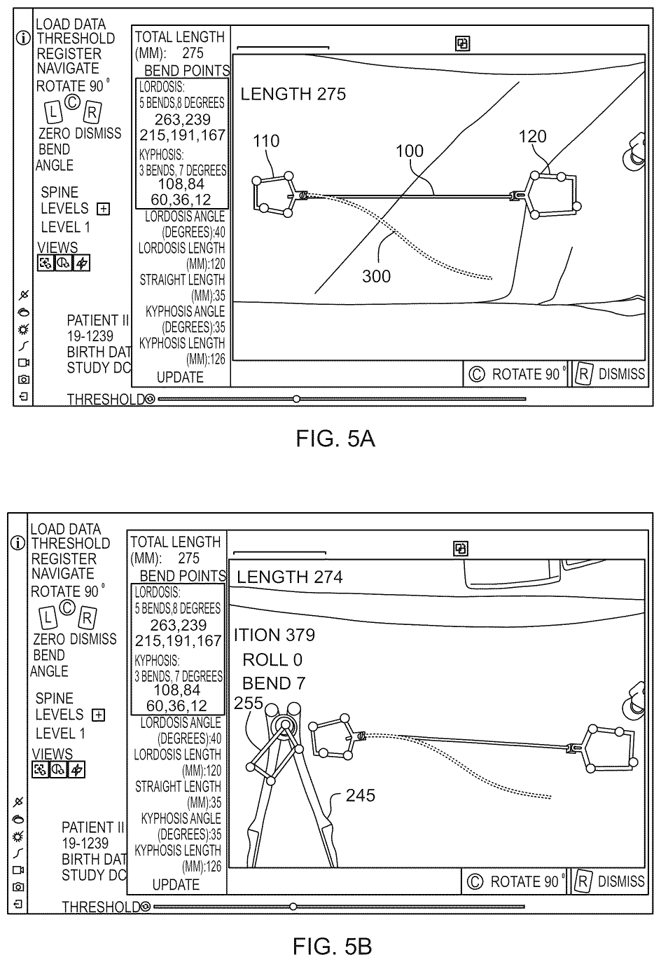

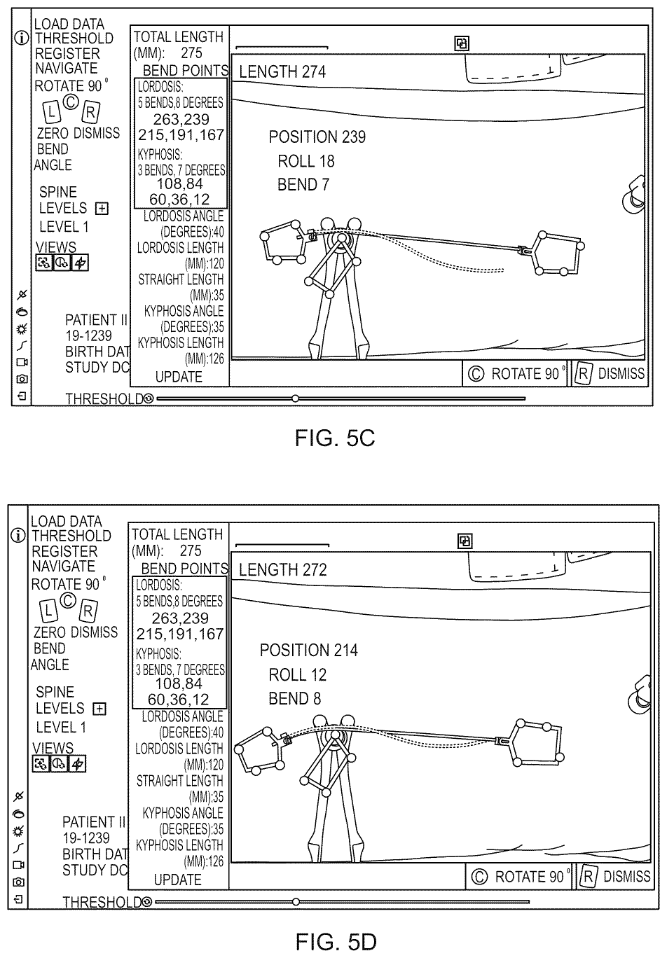

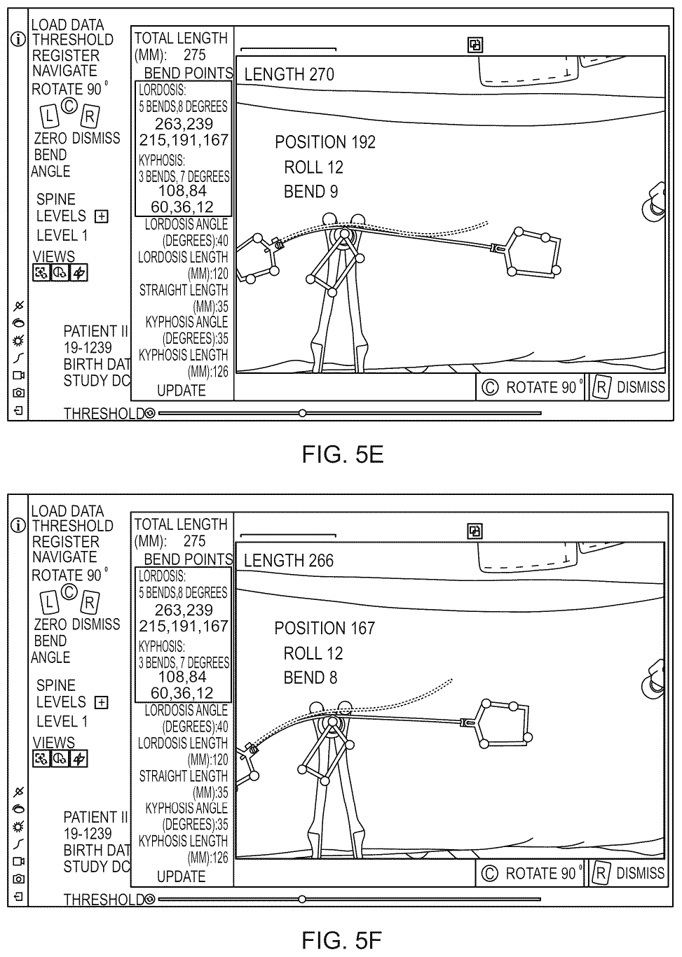

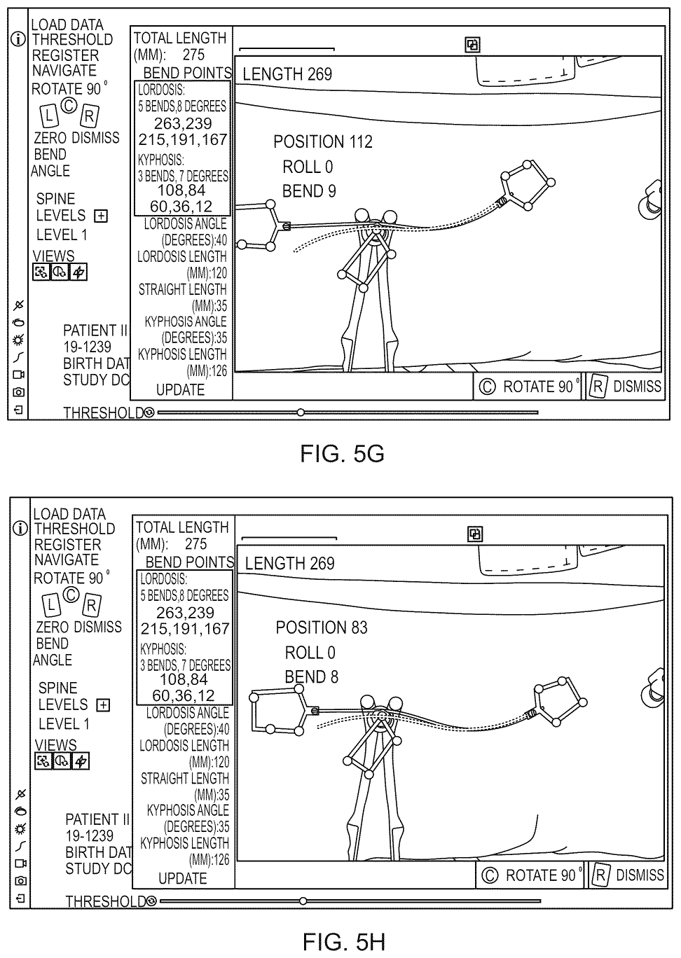

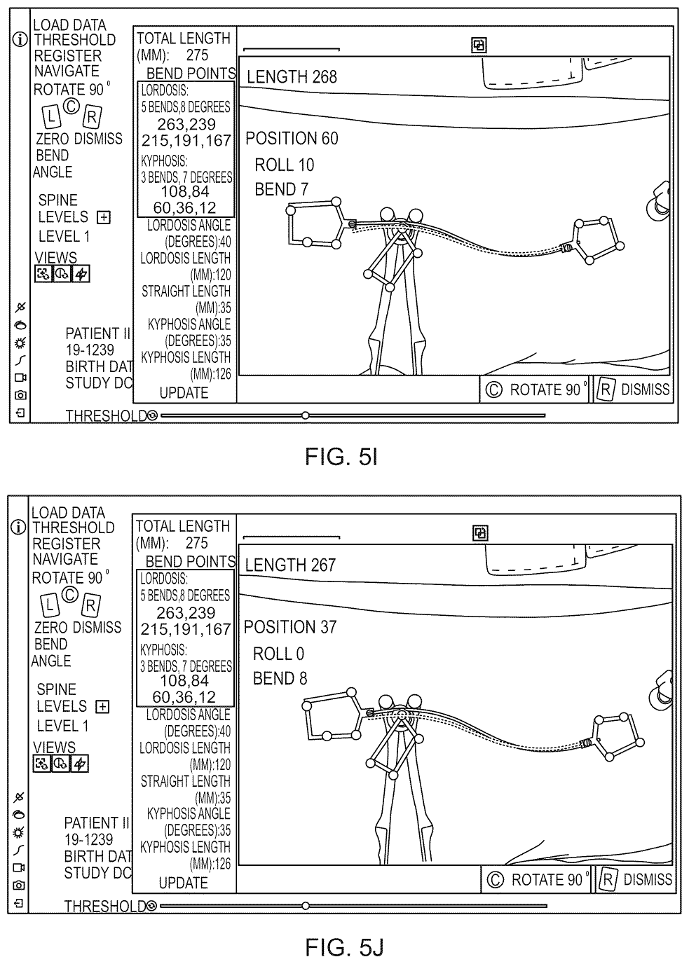

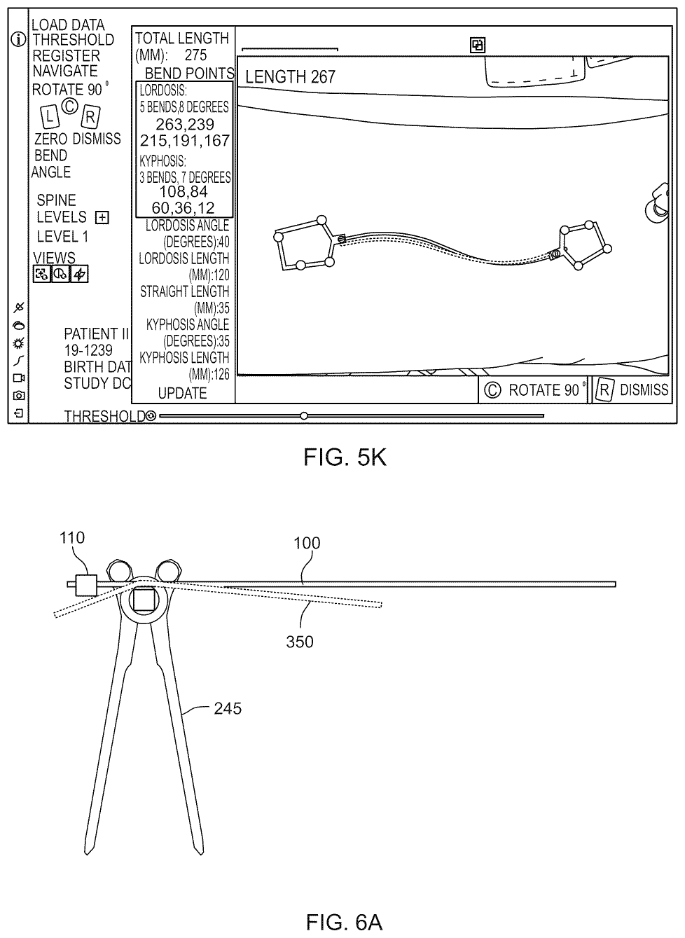

An example implementation of an augmented reality embodiment is shown in FIGS. 5A-5K, which show an example user interface that displays an augmented reality image of a surgical rod 100 having first and second trackable reference frames 110, 120, the augmented reality image including an augmented reality image annotation in the form of an augmented reality overlay of the target rod contour 300.

In this example implementation, a set of sagittal plane bending instructions are provided in the user interface in addition to the augmented reality overlay 300 of the target rod contour. The distance between the left and right trackable reference frames 110, 120 is also shown. The augmented reality images are displayed as a video feed that is updated in real time.

In the present example implementation shown in FIGS. 5A-5K, a first end of the target rod contour augmented reality overlay is fixed (pinned) to the position of the first trackable reference frame 110 (on the left side of the surgical rod 100) such that the initial slope of the target rod contour overlay matches the local slope of the actual surgical rod 100 in the region local to the point at which the first reference frame 110 is secured on the surgical rod 100. In other words, the initial portion of the target rod contour overlay is tangentially aligned with the real surgical rod 100 at the location of the first trackable reference frame 100. As shown in FIG. 5A, the other end of the target rod contour overlay is held at a constant position relative to the first trackable reference frame during the bending process. As a consequence, as shown in FIGS. 5B-5K, the sequential formation of each bend causes the shape of the real surgical rod 100 to converge to that of the target rod contour overlay 300, with the target rod contour 300 overlapping with the final bent real surgical rod 100 in the final image shown in FIG. 5K.

In the present example implementation, the target rod shape was specified by the following example parameters: lordosis angle, lordosis length, straight length, kyphosis angle and kyphosis length. Employing these parameters and assuming a constant radius of curvature and an example 5-point bend over both the kyphotic (35/5=7 degrees per bend) and lordotic (40/5=8 degrees per bend) part of the target rod contour, bend points are calculated as shown.

The bend points were shown relative to the second trackable reference frame 120 residing on the right side of the surgical rod 100, such that as the operator performs the bends unidirectionally starting from the leftmost bend residing closest to the first trackable reference frame 110 and ending the rightmost bend residing closest to the second trackable reference frame 120, the prescribed bend locations remain constant and accurate without the need to perform complex calculations and modelling of the evolving rod shape.

As shown in FIG. 5B, the rod bender may include an additional trackable reference frame 255, which permits measurement of the roll of the rod relative to the rod bender, which can be employed to provide roll angle feedback enabling each bends in to be formed in a desired plane (e.g. all bends being made in a single plane). For example, during the formation of the bends shown in FIGS. 5A-5K according to the guidance provided via the augmented reality overlay, the bends were formed by rotating the rod bender to a roll value of zero (or any other constant value). Because of the elastic nature of the surgical rod, the operator over-bent the surgical rod in order to achieve the prescribed bend angle. It is noted that roll values shown in FIGS. 5A-5K are non-zero because the example French bender was disengaged from the rod to allow full relaxation.

However, as noted above, in other example implementations that do not employ unidirectional bend formation, the changing shape of the rod as bends are formed may be modeled based on the measured bend angles, the relative roll of the rod is when each bend is executed, and known bending properties of the rod bender (e.g. the radius of curvature of a French bender), which enables the prescribed bend locations and prescribed bend angles to be updated after each bend, allowing the operator to execute the bends in any order.

In will be understood that additional augmented reality overlays may be provided in addition to the to target rod shape augmented reality overlay. For example, if the tracking system is employed to track the position of the rod bender, the tracked position the rod bender and the tracked position and orientation of at least one of the first trackable reference frame and the second trackable reference frame may be employed to the augmented reality images with positioning feedback suitable for determining when the rod bender is positioned at each prescribed bend location. For example, an augmented reality overlay may be provided showing the correct location of the rod bender for forming a given bend, and optionally showing the correct orientation of the rod bender for forming a given bend at a prescribed roll angle.

In some example embodiments in which a tracking system is employed to track a position and an orientation of the rod bender, and where at least one trackable reference frame is secured to the surgical rod, augmented reality images may be generated that facilitate the formation of bends at the prescribed bend locations and with the prescribed bend angles by including, in the augmented reality images, an augmented reality overly pinned to the tracked position of the rod bender, where the pinned augmented reality overly provides a visual indication of the prescribed bend profile.

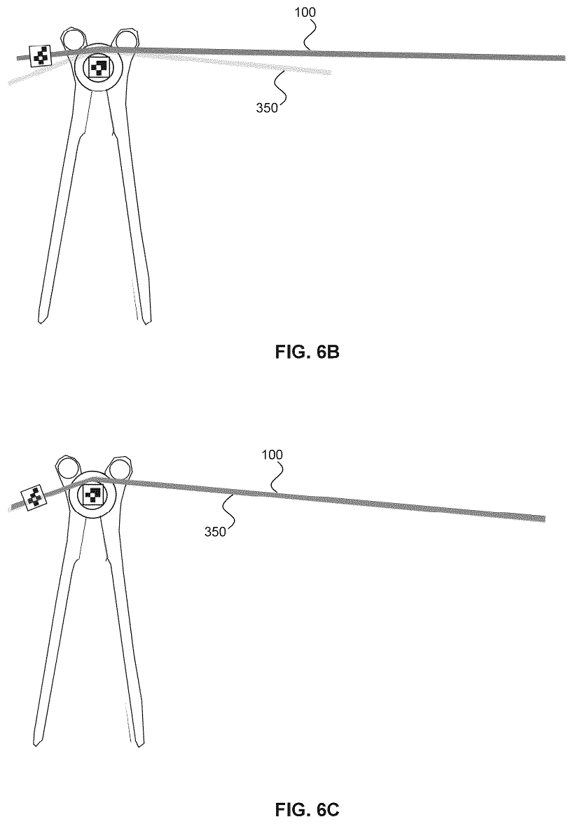

An example implementation of such an embodiment is shown in FIGS. 6A-6C, in which an augmented reality overlay 350 is shown pinned to the tracked rod bender 245, such that the augmented reality overlay 350 shows, in the form of linear segments that extend from either side of the rod bender and indicate the straight profiles of the surgical rod after it has been bent to the correct prescribed angle for a given bend. As shown in FIGS. 6B and 6C, when the tracked rod bender 245 is positioned at the prescribed position of a given bend, and the augmented reality overlay 350 facilitates a determination of when the prescribed bend has been achieved.

Notably, in the present example implementation, the augmented reality overlay 350 does not show the full target rod contour, and instead only provides an indication of the prescribed rod profile in a region proximal to the prescribed bend. For example, and generated such that after the surgical rod is bent to the prescribed bend angle associated with the bend, the surgical rod spatially overlaps with the augmented reality image annotation within at least a region proximal to the bend. In other example implementations, the augmented reality overlay that is pinned to the rod bender 245 may additionally or alternatively indicate the desired curved profile that is generated by the action of the bender. In general, the augmented reality overlay (image annotation) is pinned to the tracked position of the rod bender and generated such that after the surgical rod is bent to the prescribed bend angle associated with the bend, the surgical rod spatially overlaps with the augmented reality image annotation within at least a region proximal to the bend.

In some example implementations involving the generation of an augmented reality overlay that is fixed to the location of the tracked rod bender, the positioning information may be provided sequentially for forming the bends unidirectionally from an initial bend location that is furthest from the first location associated with the trackable reference frame 110 secured to the surgical rod, with each prescribed bend location being determined along an unbent portion of the surgical rod extending to the first location. In other example implementations in which the bends are not formed unidirectionally, a second trackable reference frame can be secured to the surgical rod and tracked with the tracking system, thereby facilitating the updating of the prescribed bend locations for bends formed between two previously formed bends, using the known bending characteristics of the rod bender, as explained above.

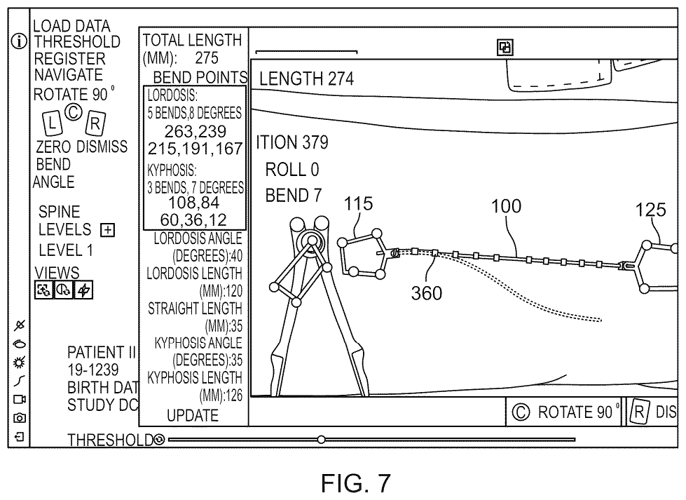

In some example embodiments in which a trackable reference frame is secured to the surgical rod, such that the surgical rod, in an initial unbent state, extends from the location of the trackable reference frame along a rod axis, augmented reality images can be generated with bend location augmented reality overlays in that indicate the prescribed bend locations along the rod axis. An example implementation of the present embodiment is illustrated in FIG. 7, in which bend location augmented reality overlays 300 are shown extending, from a first trackable reference frame 125 along the unbent portion of the surgical rod 100. While the figure shows a second trackable reference frame 115, the presence of the second trackable reference frame 115 is not necessary to generate the aforementioned bend location augmented reality overlays.

As can be seen from the figure, when the bends are formed in a unidirectional sequence of bends beginning from a bend that lies at a furthest distal location relative to the trackable reference frame, the bend location overlays 360 that lie on the proximal side of a given bend show the locations along the remaining unbent portion of the surgical rod for forming the remaining bends. Accordingly, as the augmented reality images are updated during bending of the surgical rod, based on the tracked position and orientation of the trackable reference frame, augmented-reality-based visual guidance is provided for bending the surgical rod according to the target rod contour.

If the second trackable reference frame 115 is included, then this second trackable reference frame can be employed, for example, to pin the location of an augmented reality overlay showing the final rod contour, as shown in FIG. 7. As shown in the figure and as described above, the augmented reality images may be generated such that the first end of the target rod contour is tangentially aligned with the surgical rod at the location of the second trackable reference frame 115.

While the preceding example embodiments provide guidance for rod manipulation based on a parametrization of a target rod profile as a series of bends that are to be performed at prescribed bend locations with associated prescribed bend angles, some example embodiments of the present disclosure employ augmented reality images to provide guidance for the manipulation of a surgical rod in the absence of providing guidance for bending the rod at specific bend locations.

In one such example embodiment, a tracking system is employed to track the position and orientation of a trackable reference frame secured to a surgical rod, for example, at or near one end of the surgical rod. The tracked position and orientation of the trackable reference frame, and a known coordinate transformation between the tracking system and the augmented reality display device, are employed to generate real-time augmented reality images that include an augmented reality overlay indicating the target rod contour. The target rod contour overlay is displayed such that a first end of the target rod contour is pinned to location of the trackable reference frame and the orientation of the target rod overlay is determined based on the orientation of the first trackable reference frame. The operator employs the target rod contour as a visual guide when forming bends along the surgical rod, with the target rod contour providing visual feedback of how close the shape of the real surgical rod is to the target rod contour.

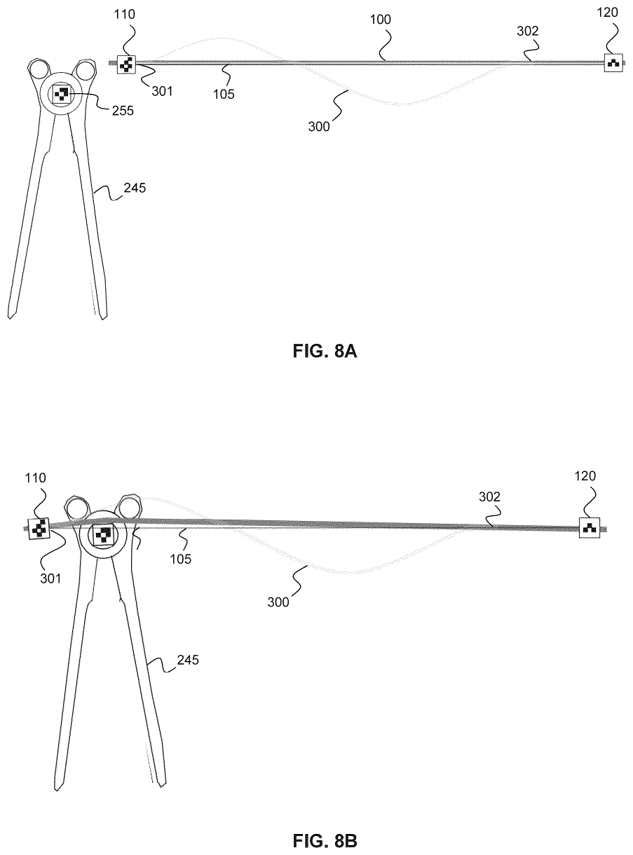

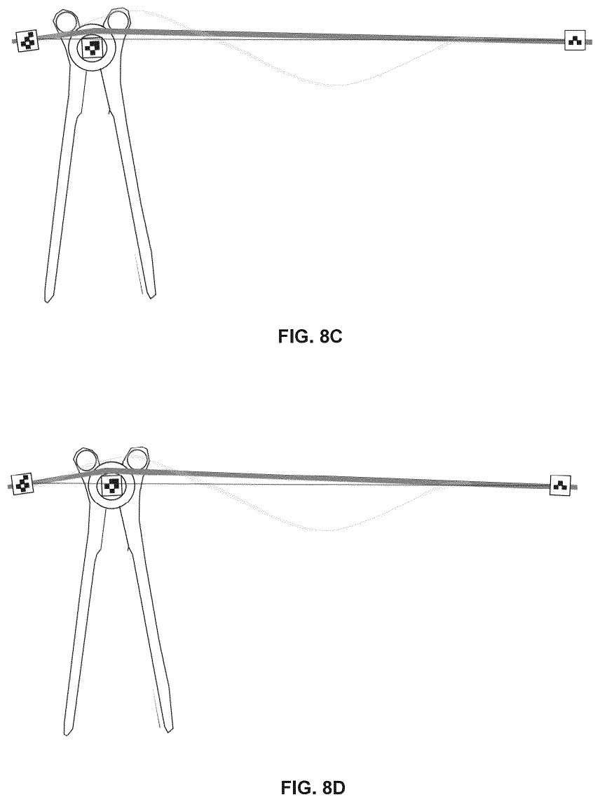

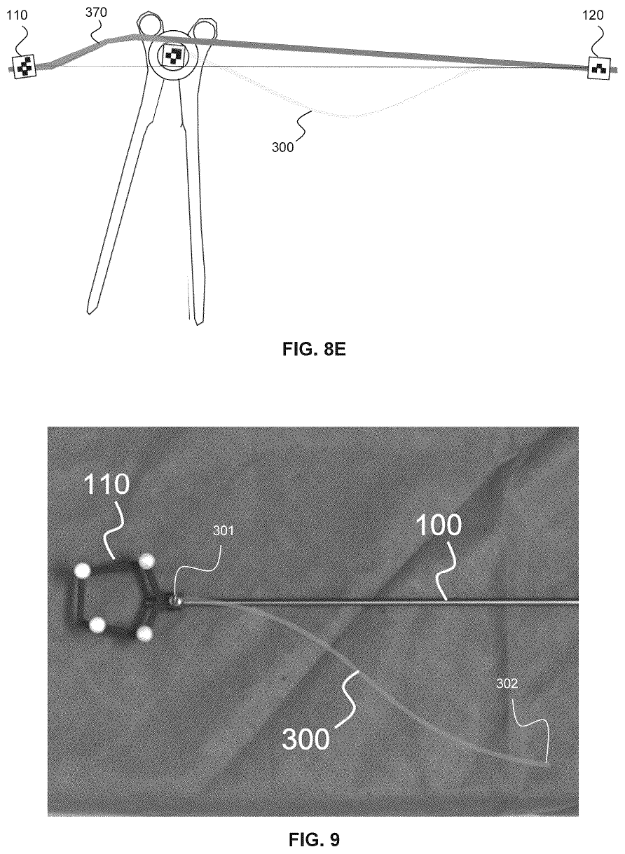

FIGS. 8A-8E illustrate an example implementation of an example implementation in which the target rod overlay 300 is generated such that a first end 301 of the target rod overlay 300 is pinned to the location of a first trackable marker 110 and a second end 302 of the target rod overlay 300 is constrained to lie along a line 105 connecting the first trackable marker 110 and the second trackable marker 120. The target rod overlay 300 provides a visual guide for manipulating the rod shape to replicate or approximate the desired target rod contour via the rod bender 245 (which may be tracked via a trackable reference frame 255).

As shown in FIGS. 8B-8E, as the rod bender 245 is employed to bend the surgical rod 100, the first end 301 of the target rod overlay 300 remains pinned to the location of the first trackable marker 110 and the second end 302 of the target rod overlay 300 remains constrained to lie along the line 105 connecting the first trackable marker 110 and the second trackable marker 120. As a result, the orientation of the target rod overlay 300 changes during the formation of the bends, but the constrained orientation of the target rod overlay 300 ensures that the target rod overlay 300 is not re-oriented at wide angles relative to the evolving shape of the surgical rod 100 during the bending process. FIGS. 8B-8E illustrate the shaping of the portion of the surgical rod to match the initial curved portion of the target rod contour 300. While not shown in FIGS. 8A-8E, the tracked orientations of the first and second trackable reference frames 110, 120 may be employed to provide bend angle feedback indicative of the angle formed by the rod bender during the formation of a given bend, using the example methods described above.

The preceding example implementation in which augmented reality guidance is provided based on constraining of the second end of the target rod overlay along a line joining the first trackable reference frame 110 and the second trackable reference frame 120 may be employed in cases in which the target rod contour lies within a single plane, with roll angle guidance optionally being provided based on the tracked orientation of the rod bender 245. In cases in which the target rod contour involves bends that do not lie within a single plane, the guidance may be provided sequentially, with an initial target rod overlay being presented for bending the surgical rod within one plane, and a second target rod overlay being subsequently displayed that facilitates the bending of the surgical rod in a second plane, with the tracked orientation of the rod bender being employed to provide feedback suitable for controlling the roll angle of the rod bender to ensure that the manipulations are formed in the appropriate planes. In such a case, for each of the first and second target rod overlays, the second end of the target rod overlay is constrained to lie within a plane that includes the first and second trackable reference frames 110 and 120 and is oriented perpendicular to the plane in which the bends are being generated.

In another example implementation in which augmented reality guidance is provided in the absence of prescribed bend locations and prescribed bend angles is shown in FIG. 9, in which a first end 301 of the target rod contour augmented reality overlay 300 is fixed (pinned) to the position of a trackable reference frame 110 secured to the surgical rod 110 such that the initial slope of the target rod contour overlay 300 matches the local slope of the actual surgical rod 100 in the region local to the point at which the trackable reference frame 110 is secured on the surgical rod. In other words, the initial portion of the target rod contour 300 overlay is tangentially aligned with the real surgical rod 300 at the location of the trackable reference frame 110. The other end of the target rod contour overlay 302 is held at a constant position relative to the trackable reference frame 110 during the bending process. A second trackable reference frame may optionally be secured to the surgical rod 100 at a location remote from the first trackable reference frame 100, for example, in order to provide bend angle feedback and/or measure a roll angle of the surgical rod during or after bending.

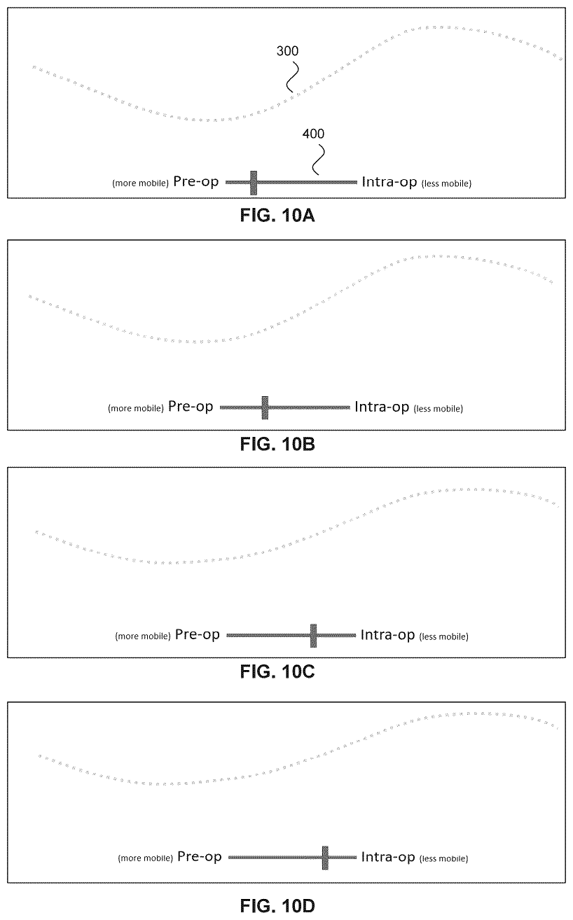

As noted above, while the target rod contour may be defined pre-operatively, some example implementations, the target rod contour may be intra-operatively refined, for example, in order to take into account differences between pre-operative and intraoperative spine configuration, and/or to account for limited mobility and flexibility that is observed intraoperatively. FIGS. 10A-10D illustrate an example user interface that facilitates the selection of a target rod contour that resides between a pre-operatively defined target rod contour and an intra-operatively measured spinal profile. The operator may employ such a user interface to change modify the target rod contour such that it becomes either closer to the preoperative plan or the intraoperative position, for example, according to an intraoperative physiological assessment of the patient mobility/stiffness.

The intraoperatively the spine profile or position may be characterized by several methods. For example, the spine profile may be characterized by touching the heads of implanted screws to characterize the left and right sides of the spine. Additionally or alternatively, surface mapping (e.g. structured light imaging) may also be used to characterize the spine by imaging and by manually or automatically selecting points (via a GUI for manual selection) that allow characterization (facet joints, screw heads) of the left and right side of the spine. Surface mapping may also be used with segmental registration to preoperative or intraoperative imaging may be used to fully characterize the 3D shape of the spine.

The example user interface shown in the figure permits the qualitative selection of an intermediate target rod contour via control of a slider that provides continuous and/or discrete variation in the target rod contour, with the modified target rod contour shape displayed to the operator.

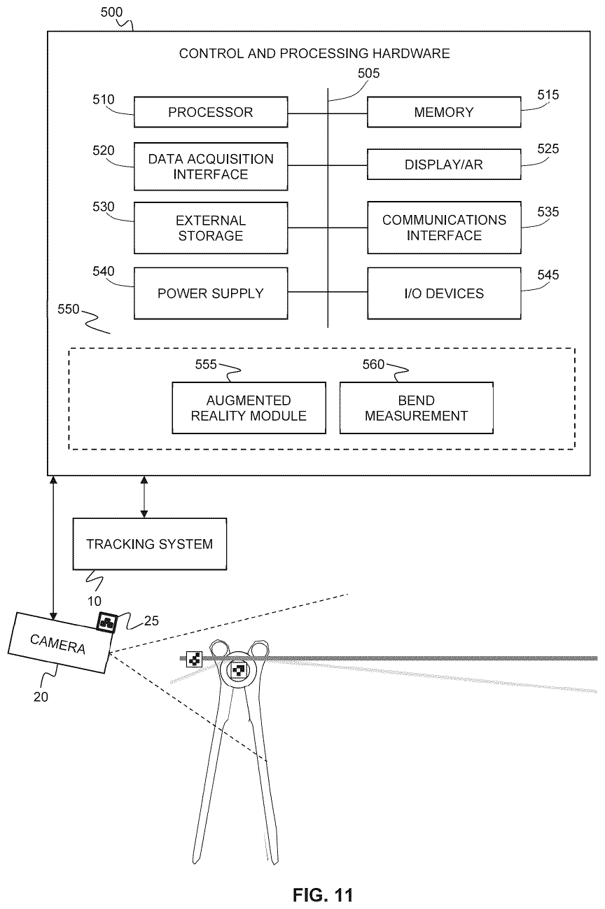

Referring now to FIG. 11, an example system is shown for providing guidance to support the bending of a surgical rod according to a target rod contour. The example system includes a tracking system 10, which may be employed to track the position and orientation of one or more trackable reference frames or single tracking markers. The system may also include a camera 20 in order to obtain images for the generation of augmented reality images. The camera 20 may optionally be trackable by the tracking system 10, e.g. via trackable reference frame 25.

In one example embodiment, the tracking subsystem 10 may include stereo cameras with integrated infrared illumination. Due to their high reflectivity to infrared light, the fiducial markers can be easily localized in each image of the two cameras. These image positions can be employed to calculate the three-dimensional position of fiducial markers by geometrical triangulation. The triangulation process can be performed, for example, by first calculating the center of mass of each of the detected markers in both camera views of the stereo calibrated camera system. This yields a set of marker points in both camera views from which the disparity between corresponding points in both views can then be calculated. This disparity along with the x and y pixel locations of each marker in one of the camera views can then be transformed into a three-dimensional spatial coordinate (in a coordinate system of the tracking system 10) using a perspective transformation. If at least three fiducial markers are rigidly attached to medical instrument 40, it is possible to compute its position and orientation (the six degrees of freedom). In alternative examples, markers such as two-dimensional barcodes or glyphs may be detected for position and orientation tracking.

While FIG. 11 illustrates an example system having two subsystems (tracking system 10 and camera 20), it is noted that alternative system configurations may be employed to perform simultaneous tracking and acquisition of images using an integrated system.

FIG. 11 also illustrates an example implementation of control and processing hardware 500, which includes one or more processors 510 (for example, a CPU/microprocessor), bus 505, memory 515, which may include random access memory (RAM) and/or read only memory (ROM), a data acquisition interface 520, a display 525 (e.g. a display screen or a heads-up display), external storage 530, one more communications interfaces 535, a power supply 540, and one or more input/output devices and/or interfaces 545 (e.g. a speaker, a user input device, such as a keyboard, a keypad, a mouse, a position tracked stylus, a position tracked probe, a foot switch, and/or a microphone for capturing speech commands).

It is to be understood that the example system shown in FIG. 11 is illustrative of a non-limiting example embodiment and is not intended to be limited to the components shown. Furthermore, one or more components of the control and processing hardware 500 may be provided as an external component that is interfaced to a processing device. For example, the tracking system 10 may be included as a component of control and processing hardware 500, or may be provided as one or more external devices.

Although only one of each component is illustrated in FIG. 11, any number of each component can be included in the control and processing hardware 500. For example, a computer typically contains a number of different data storage media. Furthermore, although bus 505 is depicted as a single connection between all of the components, it will be appreciated that the bus 505 may represent one or more circuits, devices or communication channels which link two or more of the components. For example, in personal computers, bus 505 often includes or is a motherboard. Control and processing hardware 500 may include many more or less components than those shown.

Control and processing hardware 500 may be implemented as one or more physical devices that are coupled to processor 510 through one of more communications channels or interfaces. For example, control and processing hardware 500 can be implemented using application specific integrated circuits (ASICs). Alternatively, control and processing hardware 500 can be implemented as a combination of hardware and software, where the software is loaded into the processor from the memory or over a network connection.

Some aspects of the present disclosure can be embodied, at least in part, in software. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. In general, a machine-readable medium includes any mechanism that provides (i.e., stores and/or transmits) information in a form accessible by a machine (e.g., a computer, network device, personal digital assistant, manufacturing tool, any device with a set of one or more processors, etc.).

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like. As used herein, the phrases “computer readable material” and “computer readable storage medium” refer to all computer-readable media, except for a transitory propagating signal per se.

Embodiments of the present disclosure can be implemented via processor 510 and/or memory 515. For example, the functionalities described below can be partially implemented via hardware logic in processor 510 and partially using the instructions stored in memory 515. Some embodiments are implemented using processor 510 without additional instructions stored in memory 515. Some embodiments are implemented using the instructions stored in memory 515 for execution by one or more microprocessors, which may be general purpose processors or specialty purpose processors. Thus, the disclosure is not limited to a specific configuration of hardware and/or software.

The control and processing hardware 500 is programmed with subroutines, applications or modules 550, that include executable instructions, which when executed by the one or more processors 510, causes the system to perform one or more methods described in the present disclosure. Such instructions may be stored, for example, in memory 515 and/or other internal storage.

For example, as described in detail above, the augmented reality module 555 of the control and processing hardware 100 may be employed to generate augmented reality overlays as described above and the bend measurement module 560 may be employed to measure a bend angle based on the relative change in the orientation of first and second trackable reference frames.

While many of the preceding example embodiments have been illustrated according to an example implementations in which the initial shape of the surgical rod is straight, it will be understood that many of the preceding example embodiments may be implemented and/or adapted to cases in which the surgical rod is provided in a pre-bend configuration. Moreover, it will be understood that the process of applying a bend to a surgical rod, as per bend instructions involving a bend location and a prescribed bend angle, may result in the formation of a bend or may result in the straightening out of an existing bend.

Although the preceding example embodiments provide guidance for the formation of bends in a surgical rod, it will be understood that the present example embodiments may be employed and/or adapted to provide guidance for the general shape modification or manipulation of a medical device, such as applying controlled deformations at prescribed locations in order to customize the shape of a medical device. Moreover, while the present example embodiments have referred to surgical rods, the present example embodiments may be employed and/or adapted to provide guidance for the shape modification or manipulation of a wide variety of medical devices, including, but not limited to, surgical implants, catheters, orthoses, and prostheses.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method of providing guidance for positioning and actuating a rod bender to bend a surgical rod according to a target rod contour, the method comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track:
an orientation of a first trackable reference frame secured to the surgical rod at a first location on the surgical rod; and
an orientation of a second trackable reference frame secured to the surgical rod at a second location, the second location being remote from the first location such that the prescribed bend locations reside between the first location and the second location on the surgical rod; and
providing guidance for forming at least one bend of the set of bends by:
employing the tracked orientation of the first trackable reference frame and the orientation of the second trackable reference frame to determine a measured bend angle of the surgical rod during or after bending of the surgical rod by the rod bender; and
employing the measured bend angle to provide bend angle feedback suitable for determining when the surgical rod has been bent to the prescribed bend angle associated with the bend.

2. The method according to claim 1 wherein the positioning information is provided sequentially for forming the bends unidirectionally between the first location and the second location, and wherein each prescribed bend location is determined along an unbent portion of the surgical rod extending to the second location.

3. The method according to claim 1 wherein the positioning information is provided in a manner that facilitates the formation of the bends in an operator-selectable order, and wherein known bending characteristics of the rod bender are employed to determine a suitable bend location for an intermediate bend that is formed between two previously formed bends.

4. The method according to claim 1 wherein the rod bender is trackable by the tracking system, and wherein the positioning information is provided by:
employing the tracking system to track a position of the rod bender; and
employing the tracked position the rod bender and the tracked position and orientation of the first trackable reference frame and the second trackable reference frame to provide positioning feedback suitable for determining when the rod bender is positioned at the prescribed bend location.

5. The method according to claim 1 wherein a third trackable reference frame is secured to the rod bender, and wherein the bend parameters comprise prescribed roll angles corresponding to the set of bends, the method further comprising:
employing the tracking system to detect a roll angle of the rod bender; and
providing guidance for orienting the rod bender relative to the surgical rod to form at least one bend of the set of bends at a prescribed roll angle associated with the bend by:
employing the roll angle of the rod bender and the orientation of one or both of the first trackable reference frame and the second trackable reference frame to provide roll angle feedback suitable for determining when the surgical rod has been oriented to form a bend at the prescribed roll angle associated with the bend.

6. The method according to claim 1 further comprising:
employing the tracked position and orientation of the first trackable reference frame to generate real-time augmented reality images comprising a target rod contour having a first end pinned to the first location and an orientation determined based on the first trackable reference frame;
displaying the augmented reality images; and
updating the augmented reality images, during bending of the surgical rod, based on the tracked position and orientation of the first trackable reference frame, thereby providing augmented-reality-based visual guidance for bending the surgical rod according to the target rod contour.

7. The method according to claim 6 wherein the augmented reality images are generated such that the first end of the target rod contour is tangentially aligned with the surgical rod.

8. The method according to claim 6 wherein the rod bender is trackable by the tracking system, and wherein the positioning information is provided by:
employing the tracking system to track a position of the rod bender; and
employing the tracked position the rod bender and the tracked position and orientation of the first trackable reference frame and the second trackable reference frame to annotate the augmented reality images with positioning feedback suitable for determining when the rod bender is positioned at each prescribed bend location.

9. The method according to claim 8 wherein the positioning feedback comprises generating, in the augmented reality image, an image annotation indicating a prescribed location of the rod bender for forming at least one the bends.

10. The method according to claim 6 further comprising employing the tracking system to track an orientation of the rod bender, and wherein the bend parameters comprise prescribed roll angles corresponding to the set of bends; and
at each of the prescribed bend locations, employing the tracked orientation the rod bender and a detected orientation of one or both of the first trackable reference frame and the second trackable reference frame to provide orientation feedback suitable for determining when the rod bender is oriented according to a prescribed roll angle.

11. A method of providing guidance for positioning and actuating a rod bender to bend a surgical rod according to a target rod contour, the method comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod;
employing the tracking system to track a position and an orientation of the rod bender; and
providing guidance for forming at least one bend of the set of bends by:
employing the tracked position and orientation of the rod bender to generate and display real-time augmented reality images comprising an augmented reality image annotation, the augmented reality image annotation being pinned to the tracked position of the rod bender and generated such that after the surgical rod is bent to the prescribed bend angle associated with the bend, the surgical rod spatially overlaps with the augmented reality image annotation within at least a region proximal to the bend.

12. The method according to claim 11 wherein the positioning information is provided sequentially for forming the bends unidirectionally from an initial bend location that is furthest from the first location, and wherein each prescribed bend location is determined along an unbent portion of the surgical rod extending to the first location.

13. A method of providing guidance for positioning and actuating a rod bender to bend a surgical rod according to a target rod contour, the method comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod, the surgical rod extending from the first location along a rod axis;
employing the tracked position and orientation of the first trackable reference frame to generate and display real-time augmented reality images, the real-time augmented reality images comprising augmented reality image annotations indicating the prescribed bend locations along the rod axis, such that when the bends are formed in a unidirectional sequence of bends beginning from a bend that lies at a furthest distal location relative to the first trackable reference frame, the augmented reality image annotations on a proximal side of a given bend show the locations along the remaining unbent portion of the surgical rod for forming the remaining bends; and
updating the augmented reality images, during bending of the surgical rod, based on the tracked position and orientation of the first trackable reference frame, thereby providing augmented-reality-based visual guidance for bending the surgical rod according to the target rod contour.

14. The method according to claim 13 further comprising:
employing a tracking system to track an orientation of a second trackable reference frame secured to the surgical rod at a second location, the second location being remote from the first location such that the prescribed bend location resides between the first location and the second location on the surgical rod;
employing the tracked position and orientation of the second trackable reference frame to annotate the real-time augmented reality images with a target rod contour having a first end pinned to the second location and an orientation determined based on the second trackable reference frame;
updating the augmented reality images, during bending of the surgical rod, based on the tracked position and orientation of the second trackable reference frame, thereby providing augmented-reality-based visual guidance for bending the surgical rod according to the target rod contour.

15. The method according to claim 14 wherein the augmented reality images are generated such that the first end of the target rod contour is tangentially aligned with the surgical rod.

16. A method of providing guidance for positioning and actuating a rod bender to bend a surgical rod according to a prescribed bend angle at a prescribed bend location, the method comprising:
providing positioning information suitable for positioning the rod bender at the prescribed bend location along the surgical rod;
employing a tracking system to track:
an orientation of a first trackable reference frame secured to the surgical rod at a first location on the surgical rod; and
an orientation of a second trackable reference frame secured to the surgical rod at a second location, the second location being remote from the first location such that the prescribed bend location resides between the first location and the second location on the surgical rod; and
employing the tracked orientation of the first trackable reference frame and the orientation of the second trackable reference frame to determine a measured bend angle of the surgical rod during or after bending of the surgical rod by the rod bender; and
employing the measured bend angle to provide bend angle feedback suitable for determining when the surgical rod has been bent to the prescribed bend angle.

17. The method according to claim 16 wherein the bend angle feedback comprises:
providing qualitative feedback of a deviation of the measured bend angle from the prescribed bend angle.

18. The method according to claim 16 wherein the bend angle feedback comprises:
providing quantitative feedback of a deviation of the measured bend angle from the prescribed bend angle.

19. The method according to claim 16 wherein the positioning information is provided as a distance relative to at least one of the first location and the second location.

20. The method according to claim 16 wherein the rod bender is trackable by the tracking system, and wherein the positioning information is provided by:
employing the tracking system to track a position of the rod bender; and
employing the tracked position the rod bender and the tracked position and orientation of at least one of the first trackable reference frame and the second trackable reference frame to provide positioning feedback suitable for determining when the rod bender is positioned at the prescribed bend location.

21. The method according to claim 16 wherein a third trackable reference frame is secured to the rod bender, the method further comprising:
employing the tracking system to detect a roll angle of the rod bender; and
employing the roll angle of the rod bender and the orientation of one or both of the first trackable reference frame and the second trackable reference frame to provide roll angle feedback suitable for determining when the surgical rod has been bent to a prescribed roll angle.

22. A method of providing guidance for positioning and actuating a rod bender to bend a surgical rod, the method comprising:
employing a tracking system to track a position and an orientation of a first trackable reference frame secured to the surgical rod at a first location;
employing the tracked position and orientation of the first trackable reference frame to generate real-time augmented reality images comprising a target rod contour having a first end pinned to the first location and an orientation determined based on the first trackable reference frame;
displaying the augmented reality images; and
updating the augmented reality images, during bending of the surgical rod, based on the tracked position and orientation of the first trackable reference frame, thereby providing augmented-reality-based visual guidance for bending the surgical rod according to the target rod contour.

23. The method according to claim 22 wherein the augmented reality image is generated such that the first end of the target rod contour is tangentially aligned with the surgical rod.

24. The method according to claim 22 wherein a trackable marker is secured to the surgical rod at a second location remote from the first location, and wherein the augmented reality images are generated such that a second end of the target rod contour resides along line extending between the first location and the second location.

25. The method according to claim 22 wherein a second trackable reference frame is secured to the surgical rod at a second location remote from the first location, and wherein the augmented reality images are generated such that a second end of the target rod contour resides along line extending between the first location and the second location.

26. The method according to claim 25 further comprising employing the orientation of the first trackable reference frame and the orientation of the second trackable reference frame to determine a measured bend angle of the surgical rod during or after bending of the surgical rod by the rod bender; and
employing the measured bend angle to provide bend angle feedback.

27. The method according to claim 25 further comprising:
employing the tracking system to track a position and an orientation of the second trackable reference frame; and
providing feedback indicative of a roll of the second trackable reference frame relative to the first trackable reference frame.

28. The method according to claim 22 further comprising:
employing the tracking system to track a trackable rod bender having an additional trackable reference frame secured thereto and determine an orientation of the rod bender; and
providing bender orientation feedback indicative of a roll of the rod bender relative to the first trackable reference frame.

29. The method according to claim 28 wherein the target rod contour is a first target rod contour defined within a first plane, and wherein bender orientation feedback is provided to facilitate orientation of the rod bender such that a bending plane of the rod bender is parallel to the first plane during bending of the surgical rod.

30. The method according to claim 29 wherein the augmented reality images are first real-time augmented reality images, the method further comprising:
employing the position and orientation of the first trackable reference frame to generate second real-time augmented reality images comprising a second target rod contour, such that a first end of the second target rod contour is pinned to the first location, and wherein the second target rod contour resides in a second plane that is orthogonal to the first plane;
displaying the second augmented reality images; and
updating the second augmented reality images, during bending of the surgical rod in the second plane, based on the tracked position and orientation of the first trackable reference frame, thereby providing augmented-reality-based visual guidance for bending the surgical rod according to the second target rod contour;
wherein additional bender orientation feedback is provided to facilitate orientation of the rod bender such that the bending plane of the rod bender is parallel to the second plane during bending of the surgical rod.

31. The method according to claim 22 further comprising displaying, in a user interface configured to receive input for varying the target rod contour relative to a preoperatively-defined rod contour.

32. The method according to claim 31 wherein the user interface is configured such that the shape of the target rod contour variable, in response to the input, between the preoperatively-defined rod contour and an intraoperatively-determined spinal contour.

33. The method according to claim 32 wherein the user interface is configured such that the shape of the target rod contour is continuously variable, via a slider, between the preoperatively-defined rod contour and the intraoperatively-determined spinal contour.

34. A system for providing guidance for positioning and actuating a rod bender to bend a surgical rod according to a target rod contour, the system comprising:
a tracking system; and
control and processing circuitry operatively coupled to said tracking system, said control and processing circuitry comprising at least one processor and associated memory, the memory comprising instructions executable by said at least one processor for performing operations comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing said tracking system to track:
an orientation of a first trackable reference frame secured to the surgical rod at a first location on the surgical rod; and
an orientation of a second trackable reference frame secured to the surgical rod at a second location, the second location being remote from the first location such that the prescribed bend locations reside between the first location and the second location on the surgical rod; and
providing guidance for forming at least one bend of the set of bends by:
employing the tracked orientation of the first trackable reference frame and the orientation of the second trackable reference frame to determine a measured bend angle of the surgical rod during or after bending of the surgical rod by the rod bender; and
employing the measured bend angle to provide bend angle feedback suitable for determining when the surgical rod has been bent to the prescribed bend angle associated with the bend.

35. A system for providing guidance for positioning and actuating a rod bender to bend a surgical rod according to a target rod contour, the system comprising:
a tracking system; and
control and processing circuitry operatively coupled to said tracking system, said control and processing circuitry comprising at least one processor and associated memory, the memory comprising instructions executable by said at least one processor for performing operations comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing said tracking system to track an orientation of a first trackable reference frame secured to the surgical rod at a first location on the surgical rod;
employing said tracking system to track a position and an orientation of the rod bender; and
providing guidance for forming at least one bend of the set of bends by:
employing the tracked position and orientation of the rod bender to generate and display real-time augmented reality images comprising an augmented reality image annotation, the augmented reality image annotation being pinned to the tracked position of the rod bender and generated such that after the surgical rod is bent to the prescribed bend angle associated with the bend, the surgical rod spatially overlaps with the augmented reality image annotation within at least a region proximal to the bend.

36. A system for providing guidance for positioning and actuating a rod bender to bend a surgical rod according to a target rod contour, the system comprising:
a tracking system;
a display device; and
control and processing circuitry operatively coupled to said tracking system and said display device, said control and processing circuitry comprising at least one processor and associated memory, the memory comprising instructions executable by said at least one processor for performing operations comprising:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing said tracking system to track an orientation of a first trackable reference frame secured to the surgical rod at a first location on the surgical rod, the surgical rod extending from the first location along a rod axis;
employing the tracked position and orientation of the first trackable reference frame to generate and display on said display device real-time augmented reality images, the real-time augmented reality images comprising augmented reality image annotations indicating the prescribed bend locations along the rod axis, such that when the bends are formed in a unidirectional sequence of bends beginning from a bend that lies at a furthest distal location relative to the first trackable reference frame, the augmented reality image annotations on a proximal side of a given bend show the locations along the remaining unbent portion of the surgical rod for forming the remaining bends; and
updating the augmented reality images, during bending of the surgical rod, based on the tracked position and orientation of the first trackable reference frame, thereby providing augmented-reality-based visual guidance for bending the surgical rod according to the target rod contour.

37. A system for providing guidance for positioning and actuating a rod bender to bend a surgical rod according to a target rod contour, the system comprising:
a tracking system;
a display device; and
control and processing circuitry operatively coupled to said tracking system and said display device, said control and processing circuitry comprising at least one processor and associated memory, the memory comprising instructions executable by said at least one processor for performing operations comprising:
controlling said tracking system to track a position and an orientation of a first trackable reference frame secured to the surgical rod at a first location;
employing the tracked position and orientation of the first trackable reference frame to generate real-time augmented reality images comprising a target rod contour having a first end pinned to the first location and an orientation determined based on the first trackable reference frame;
displaying the augmented reality images on said display device; and
updating the augmented reality images, during bending of the surgical rod, based on the tracked position and orientation of the first trackable reference frame, thereby providing augmented-reality-based visual guidance for bending the surgical rod according to the target rod contour.

38. A non-transitory computer-readable storage medium having stored therein data representing instructions executable by a processor for providing guidance for positioning and actuating a rod bender to bend a surgical rod according to a target rod contour, the storage medium comprising instructions for performing operations including:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
controlling a tracking system to track:
an orientation of a first trackable reference frame secured to the surgical rod at a first location on the surgical rod; and
an orientation of a second trackable reference frame secured to the surgical rod at a second location, the second location being remote from the first location such that the prescribed bend locations reside between the first location and the second location on the surgical rod; and
providing guidance for forming at least one bend of the set of bends by:
employing the tracked orientation of the first trackable reference frame and the orientation of the second trackable reference frame to determine a measured bend angle of the surgical rod during or after bending of the surgical rod by the rod bender; and
employing the measured bend angle to provide bend angle feedback suitable for determining when the surgical rod has been bent to the prescribed bend angle associated with the bend.

39. A non-transitory computer-readable storage medium having stored therein data representing instructions executable by a processor for providing guidance for positioning and actuating a rod bender to bend a surgical rod according to a target rod contour, the storage medium comprising instructions for performing operations including:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod;
employing said tracking system to track a position and an orientation of the rod bender; and
providing guidance for forming at least one bend of the set of bends by:
employing the tracked position and orientation of the rod bender to generate and display real-time augmented reality images comprising an augmented reality image annotation, the augmented reality image annotation being pinned to the tracked position of the rod bender and generated such that after the surgical rod is bent to the prescribed bend angle associated with the bend, the surgical rod spatially overlaps with the augmented reality image annotation within at least a region proximal to the bend.

40. A non-transitory computer-readable storage medium having stored therein data representing instructions executable by a processor for providing guidance for positioning and actuating a rod bender to bend a surgical rod according to a target rod contour, the storage medium comprising instructions for performing operations including:
obtaining bend parameters for bending the surgical rod according to a set of bends that approximate the target rod contour, the bend parameters comprising prescribed bend locations along the surgical rod and corresponding prescribed bend angles;
providing positioning information suitable for positioning the rod bender at the prescribed bend locations along the surgical rod;
employing a tracking system to track an orientation of a first trackable reference frame secured to the surgical rod at a first location on the surgical rod, the surgical rod extending from the first location along a rod axis;
employing the tracked position and orientation of the first trackable reference frame to generate and display on a display device real-time augmented reality images, the real-time augmented reality images comprising augmented reality image annotations indicating the prescribed bend locations along the rod axis, such that when the bends are formed in a unidirectional sequence of bends beginning from a bend that lies at a furthest distal location relative to the first trackable reference frame, the augmented reality image annotations on a proximal side of a given bend show the locations along the remaining unbent portion of the surgical rod for forming the remaining bends; and
updating the augmented reality images, during bending of the surgical rod, based on the tracked position and orientation of the first trackable reference frame, thereby providing augmented-reality-based visual guidance for bending the surgical rod according to the target rod contour.

41. A non-transitory computer-readable storage medium having stored therein data representing instructions executable by a processor for providing guidance for positioning and actuating a rod bender to bend a surgical rod according to a target rod contour, the storage medium comprising instructions for performing operations including:
controlling a tracking system to track a position and an orientation of a first trackable reference frame secured to the surgical rod at a first location;
employing the tracked position and orientation of the first trackable reference frame to generate real-time augmented reality images comprising a target rod contour having a first end pinned to the first location and an orientation determined based on the first trackable reference frame;
displaying the augmented reality images on a display device; and
updating the augmented reality images, during bending of the surgical rod, based on the tracked position and orientation of the first trackable reference frame, thereby providing augmented-reality-based visual guidance for bending the surgical rod according to the target rod contour.
